(19) United States
(12) Patent Application Publication
(10) Pub. No.: US 2020/0348294 A1
(43) Pub. Date: Nov. 5, 2020

(54) HYPERCROSSLINKING WITH DIAMINE CROSSLINKERS

(71) Applicant: Roche Diagnostics Operations, Inc., Indianapolis, IN (US)

(72) Inventors: Gaston Hubertus Maria Vondenhoff, Muenchen (DE); Stephan Hug, Muenchen (DE)

(73) Assignee: Roche Diagnostics Operations, Inc., Indianapolis, IN (US)

(21) Appl. No.: 16/931,599

(22) Filed: Jul. 17, 2020

Related U.S. Application Data
(63) Continuation of application No. PCT/EP2019/051152, filed on Jan. 17, 2019.

(30) Foreign Application Priority Data
Jan. 18, 2018 (EP) 18152316.8
Oct. 17, 2018 (EP) 18200930.8

(51) Int. Cl. G01N 33/543; C08F 292/00; C08J 3/24; C08K 3/22; C08G 83/00; C08K 9/04; C08K 5/17; H01F 1/00

(52) U.S. Cl. CPC G01N 33/54326; C08F 292/00; C08J 3/242; C08K 3/22; C08G 83/005; C08K 9/04; C02F 2101/30; H01F 1/0054; C08J 2325/04; C08K 2003/2275; C08K 2003/2272; C08K 2201/01; C08K 2201/011; C08K 5/17

(57) ABSTRACT

The present disclosure relates to hypercrosslinked magnetic particle with a polymer matrix and at least one magnetic core (M), wherein the polymer matrix has at least one crosslinked polymer having at least one hypercrosslinking bond, wherein the hypercrosslinking bond is a molecule having at least two nitrogen atoms within its structure which are part of the hypercrosslinking bond; and having at least one positive charge. Further, the disclosure relates to a method of preparing the hypercrosslinked magnetic particle and also to hypercrosslinked magnetic particle obtained or obtainable from the method. Also described is the use of the hypercrosslinked magnetic particles for enrichment or purification of at least one analyte as well as to the use of the hypercrosslinked magnetic particles for purification of water.

### CROSS-REFERENCE TO RELATED APPLICATIONS

This application is a continuation of International Application No. PCT/EP2019/051152 filed Jan. 17, 2019, which claims priority to European Application Nos. 18152316.8 filed Jan. 18, 2018, and 18200930.8 filed Oct. 17, 2018, the disclosures of which are hereby incorporated by reference in their entirety.

### FIELD OF THE INVENTION

The present invention relates to a hypercrosslinked magnetic particle comprising a polymer matrix and at least one magnetic core (M), wherein the polymer matrix comprises at least one crosslinked polymer having at least one hypercrosslinking bond, wherein the hypercrosslinking bond consists of a molecule comprising at least two nitrogen atoms within its structure which are part of the hypercrosslinking bond and having at least one positive charge, wherein the molecule comprising at least two nitrogen atoms within its structure has the general structure of formula I. Further, the invention relates to a method of preparing said hypercrosslinked magnetic particle and also to a hypercrosslinked magnetic particle obtained or obtainable from said method. In a further aspect, the present invention relates to the use of the hypercrosslinked magnetic particle for qualitative and/or quantitative determination of at least one analyte in a fluid or gas. Furthermore, the invention relates to the use of the hypercrosslinked magnetic particles for enrichment or purification of at least one analyte as well as to the use of the hypercrosslinked magnetic particles for purification of water.

### BACKGROUND

Magnetic particles are a great tool for capturing analytes from human samples. When e.g. covered with antibodies, these particles are able to specifically capture analytes which can be detected by optical techniques. The magnetic properties are of great importance as they allow easy, fast and cheap automation on diagnostic systems and additionally avoid time-consuming centrifugation and filtration steps. Superparamagnetic materials get more attention as they only show magnetization when an external magnetic field is applied. In the absence of an external magnetic field, magnetization appears to be zero (no “memory effect”). Great varieties of beads are known and are commercially available. High specific surface areas on the magnetic particles are required to enrich analytes from samples. To increase the surface area to more than 300 m²/g, magnetic particles need to be coated with a porous matrix. This is usually done by embedding magnetic particles into a porous silica- or titanium oxide-matrix. One drawback is the high density of silica and titanium oxide that leads to a decrease of magnetization with increasing film thickness. Furthermore, only mesoporous (pores >2 nm) systems can be developed by using silica or titanium oxide but especially for small analytes, materials with micropores (pores <2 nm) are preferred. Additionally, proteins and phospholipids are adsorbed into the large mesopores, which generates problematic matrix-effects in the LC/MS system. Therefore, a low density porous polymer matrix (smaller loss of magnetization) with micropores should be more benefitial compared to a silica matrix. Yang et al. as well as Xu et al. describe the preparation of magnetic porous polymer particles [1,2].Geremia et al. describe a polymer comprising acidic monomers and ionic monomers connected to form a polymeric backbone [3]. Disclosed is the formation of a cross-linked shell around a core material, wherein FIG. 5A shows exemplary polymers with imidazole containing cross-linking bonds between two polymeric chains. However, the crosslinking is effected by further crosslinking molecules that react with the imidazole groups that were already attached to the polymers. Georgi et al. describes the synthesis of hyperbranched poly(4-chloromethylstyrene) and derviatization thereof via click chemistry or esterification, without crosslinking [4]. Mueller-Schulte describes the synthesis of magnetic beads with a polyvinyl alcohol (PVAL) polymer coating [5]. The type of coating should allow for further derivatization by making use of the reactive hydroxyl groups. Glutaraldehyde (a dialdehyde) is most commonly used since it reacts within a few minutes. If a dialdehyde is used as a crosslinking compound in combination with a diamine the resulting product are ketals, or in the unlikely event that the diamines be incorporated into the crosslinking moiety, then a Schiff base is formed. CN 106 432 562 A describes the preparation of magnetic chloromethylated polystyrene nanospheres, wherein the—polymers are not crosslinked [6].

Usually, polystyrene networks can be formed by crosslinking polystyrene chains or styrene-divinylbenzene copolymers with the aid of crosslinking agents or by the copolymerization of styrene units with reactive groups, which can act as internal crosslinkers [7]. Typical crosslinking agents are bis-chlorobenzyl compounds which react in the presence of Friedel-Crafts (FC) catalysts with the aromatic backbone of the styrene chains forming crosslinking bridges. For internal crosslinking, usually vinylbenzylchloride is used for formation of the copolymer and crosslinked under Friedel-Crafts conditions as well. For the hypercrosslinking reaction the polysterene polymers are typically swollen in dichloroethane and as Friedel-Crafts catalyst the Lewis acid FeCl₃ is used [8,9]. The reaction conditions are harsh with elevated temperatures (usually 80° C.), long reaction times (>16 h) and high concentrations of the Lewis acid. The main side product of the Friedel-Crafts reaction is hydrochloric acid (HCl), which is harmful for polysterene materials that contain magnetic components such as magnetite or maghemite due to dissolution effects.

In order to further modify the polystyrene networks, i.e. to further introduce functional groups that alter the chemical and physical properties of these beads, vinyl benzyl chloride may be also used as a monomer that is further derivatized by reacting the benzylic chloride.

For using the magnetic beads in diagnostic tests they have to be hydrophilic since the test material is usually based on aqueous media. To make the beads more hydrophilic, in a final step all remaining benzyl chloride moieties (i.e. those that were not consumed in the Friedel-Craft reaction) are reacted with hydroxide ions to introduce hydroxyl groups.

One drawback of the conventional Friedel-Craft reaction as mentioned-above is the formation of HCl since it causes the magnetite inside the bead to dissolve, making the beads less magnetic. Furthermore, the final hydroxylation of the “classical” beads is an extra step that indeed makes the beads more hydrophilic but does not introduce a charge. Although not a disadvantage per se, this extra step may be omitted, saving time, reducing costs and waste. In addition, the introduction of functionality in such a way that charge is introduced cannot be done by the convention method. However, this kind of functionality, i.e. a charge, would be highly advantageous for analyte capturing, especially for those analytes having opposite charge(s). A further drawback of the convention Friedel-Crafts reaction is the required catalyst FeCl₃ since this corrosive reagent is only very limited water soluble and in hydroxylized form tends to attach to storage/reaction vessels (e.g. glass or steel), making this an unpractical reagent.

The technical problem underlying the present invention was therefore the provision of magnetic beads which have charge(s) and further the provision of a method for preparing such magnetic beads having charge(s), preferably without the need to use corrosive reagents and without formation of components such as HCl which are harmful for the magnetic beads' magnetite.

### SUMMARY OF THE INVENTION

This problem is solved by the invention with the features of the independent patent claims. Advantageous developments of the invention, which can be realized individually or in combination, are presented in the dependent claims and/or in the following specification and detailed embodiments.

The present disclosure relates to hypercrosslinked magnetic particle with a polymer matrix and at least one magnetic core (M), wherein the polymer matrix has at least one crosslinked polymer having at least one hypercrosslinking bond, wherein the hypercrosslinking bond is a molecule having at least two nitrogen atoms within its structure which are part of the hypercrosslinking bond; and having at least one positive charge. Further, the disclosure relates to a method of preparing the hypercrosslinked magnetic particle and also to hypercrosslinked magnetic particle obtained or obtainable from the method. Also described is the use of the hypercrosslinked magnetic particles for enrichment or purification of at least one analyte as well as to the use of the hypercrosslinked magnetic particles for purification of water.

### BRIEF DESCRIPTION OF THE FIGURES

FIG. 1 shows the enrichment workflow for the hypercrosslinked porous magnetic polymer particles (beads).

FIG. 2 depicts analyte recoveries that were obtained after sample preparation using the enrichment workflow as illustrated in FIG. 1.

FIG. 3 shows that diamine hypercrosslinked porous magnetic polymer particles were less sensitive to the choice of organic solvent that is used for elution of the analytes from the hypercrosslinked porous magnetic polymer particles, wherein two organic solvents were chosen for this purpose: MeOH and CH₃CN. Diazepam is exemplary to most included analytes.

FIG. 4 depicts expected analyte recoveries as described in Example 3 for hypercrosslinked porous magnetic polymer particles (3a) to (3d) for the analytes gemtamicin, methotrexate, norbuprenorphin-glucuronide and tobramycin.

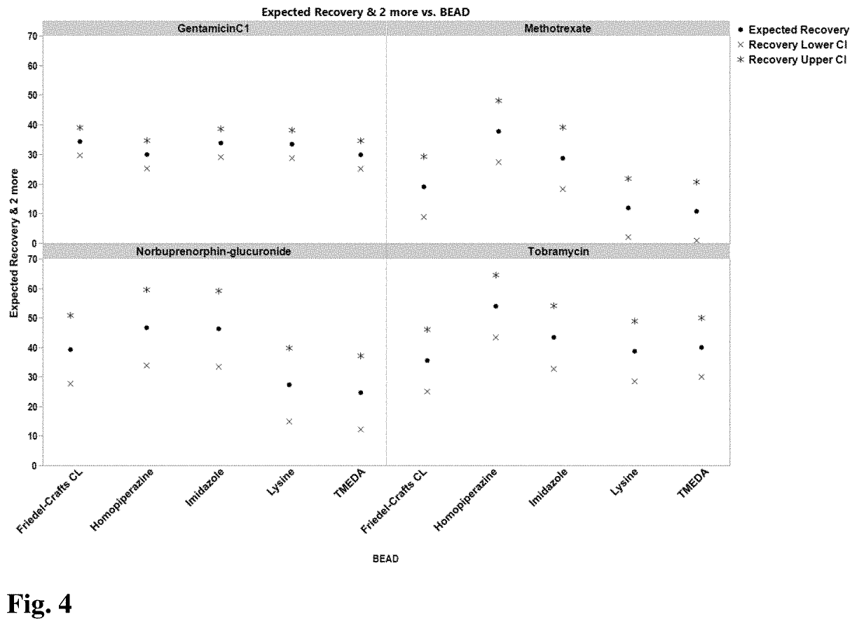

FIG. 5 depicts expected analyte recoveries as described in Example 3 for hypercrosslinked porous magnetic polymer particles (3a) to (3d) for the analytes 2-oxo-3-hydroxy-LSD, amikacin, benzoylecgonine and ethylglucuronide.

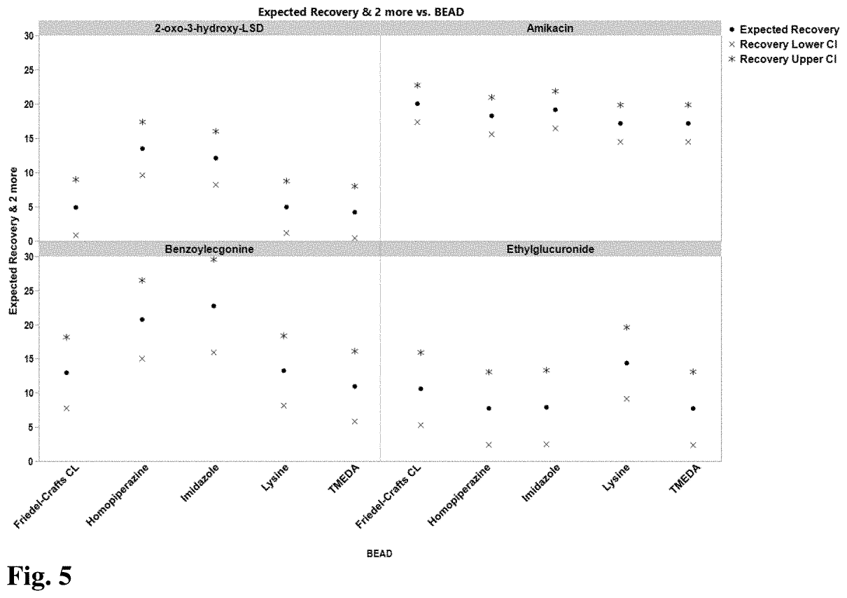

FIG. 6 depicts expected analyte recoveries as described in Example 3 for hypercrosslinked porous magnetic polymer particles (3a) to (3d) for the analytes 5-fluorouracil, exgonine, gabapentin and pregabalin.

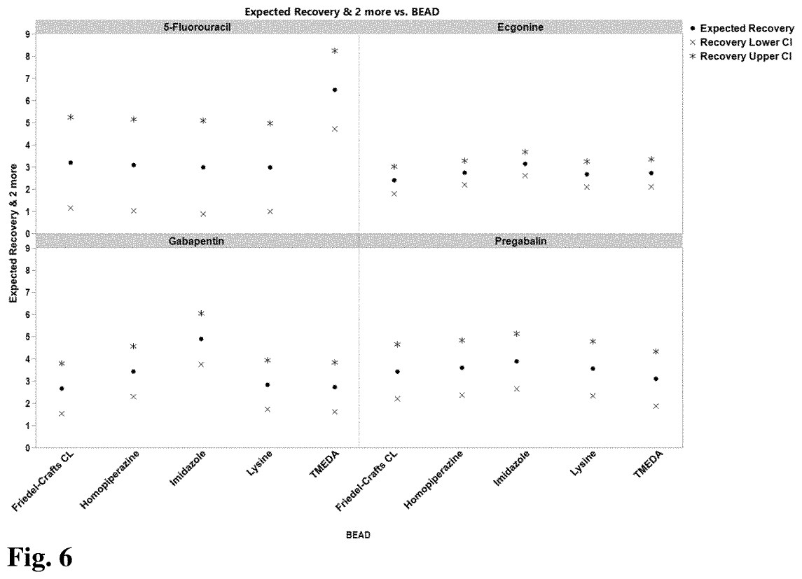

FIG. 7 depicts expected analyte recoveries as described in Example 3 for hypercrosslinked porous magnetic polymer particles (3a) to (3d) for the analytes ethylsulfate, morphine-3-glucuronide, noroxymorphone and buprenorphine-glucuronide.

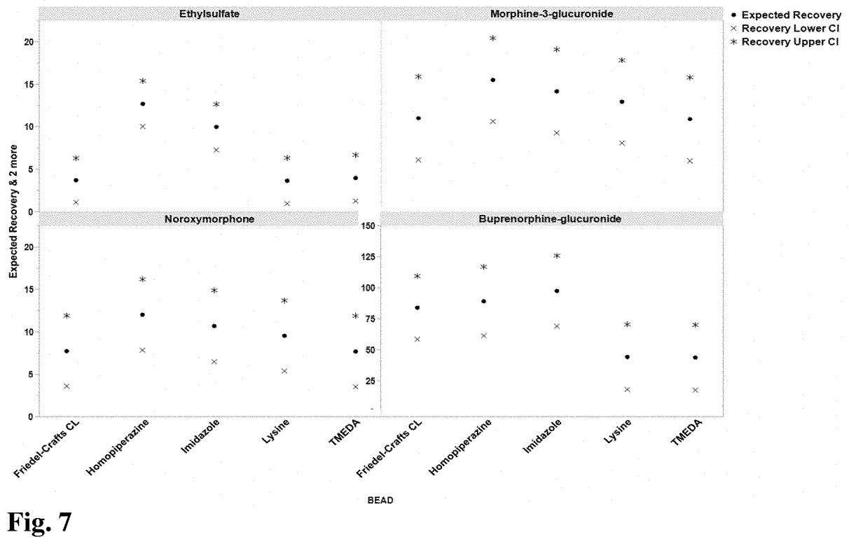

FIG. 8 shows absolute analyte recoveries for each bead, under optimal workflow conditions, purified from urine.

FIG. 9 shows absolute analyte recoveries for each bead, under optimal workflow conditions, purified from serum.

FIG. 10 shows the difference in absolute recoveries for urine analytes. (Mean recovery optimal TMEDA-bead)−(Mean recovery optimal FC-bead).

FIG. 11 shows the difference in absolute recoveries for serum analytes. (Mean recovery optimal TMEDA-bead)−(Mean recovery optimal FC-bead).

### DETAILED DESCRIPTION OF THE INVENTION

As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements.

Further, it shall be noted that the terms “at least one”, “one or more” or similar expressions indicating that a feature or element may be present once or more than once, typically will be used only once when introducing the respective feature or element. In the following, in most cases, when referring to the respective feature or element, the expressions “at least one” or “one or more” will not be repeated, notwithstanding the fact that the respective feature or element may be present once or more than once.

Further, as used in the following, the terms “preferably”, “more preferably”, “particularly”, “more particularly”, “specifically”, “more specifically” or similar terms are used in conjunction with optional features, without restricting alternative possibilities. Thus, features introduced by these terms are optional features and are not intended to restrict the scope of the claims in any way. The invention may, as the skilled person will recognize, be performed by using alternative features. Similarly, features introduced by “in an embodiment of the invention” or similar expressions are intended to be optional features, without any restriction regarding alternative embodiments of the invention, without any restrictions regarding the scope of the invention and without any restriction regarding the possibility of combining the features introduced in such a way with other optional or non-optional features of the invention.

In a first aspect, the present invention relates to hypercrosslinked magnetic particle comprising a polymer matrix (P) and at least one magnetic core (M), wherein the polymer matrix (P) comprises at least one crosslinked polymer having at least one hypercrosslinking bond, wherein the hypercrosslinking bond consists of a molecule comprising at least two nitrogen atoms within its structure which are part of the hypercrosslinking bond; wherein the molecule comprising at least two nitrogen atoms within its structure has the general structure of formula I

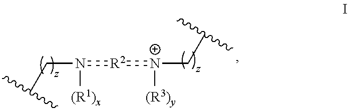

wherein
x, y are independently 1 or 2;
z is zero or 1;
R¹, R³ are independently selected from the group consisting of hydrogen, C1-C10-alkyl, C1-C10-alkenyl, C5-C10-cycloalkyl, C5-C12-aryl, C4-C10-heteroaryl and —(—O—CH₂—CH₂—)ₙ—O—CH₃ with n being an integer in the range of from 1 to 15, wherein each of R¹, R³ may have at least one further substituent selected from the group consisting of hydrogen, C1-C5-alkyl, C5-C12-aryl, C4-C10-heteroaryl, and wherein R¹ and R³ are separate or together with R² form an aliphatic or aromatic ring system;
R² is selected from the group consisting of C1-C10-alkyl, which is optionally substituted with a carboxyl(ate) group, C1-C10-alkenyl, C5-C10-cycloalkyl, C5-C12-aryl, C4-C10-heteroaryl and —(—O—CH₂—CH₂—)ₙ—O— with n being an integer in the range of from 1 to 15, wherein each cyclic structure having two or more ring systems has separated or annulated ring systems;
[symbol] the sinuous lines represent the crosslinked polymer;
   wherein the bonds connecting R² with each nitrogen atom are independently selected from the group consisting of single, double and aromatic bond; and
   wherein the molecule comprising at least two nitrogen atoms within its structure having the general structure of formula I has at least one positive charge.

According to one embodiment, the residue R² is selected from the group consisting of C1-C10-alkyl, C1-C10-alkenyl, C5-C10-cycloalkyl, C5-C12-aryl, C4-C10-hetero aryl and —(—O—CH₂—CH₂—)ₙ—O— with n being an integer in the range of from 1 to 15, wherein each cyclic structure having two or more ring systems has separated or annulated ring systems; wherein preferably each C1-C10-alkyl is not substituted with a carboxyl(ate) group, i.e. each C1-C10-alkyl has only hydrogen atoms as substituents at the carbon atoms.

The hypercrosslinked magnetic particles comprising a polymer matrix (P) and at least one magnetic core (M), wherein the polymer matrix (P) comprises at least one crosslinked polymer having at least one hypercrosslinking bond, wherein the hypercrosslinking bond comprises a molecule comprising at least two nitrogen atoms within its structure which are part of the hypercrosslinking bond; and having at least one positive charge (the diamine beads) exhibit a better performance in terms of analyte capturing and release than beads that are hypercrosslinked via the classical Friedel-Crafts alkylation route. Due to the at least one positive charge a strong anion exchange (SAX) functionality is present. Interestingly, it was found that the presence of positive charged amines is also advantageous for the recovery of analytes that are not negatively charged. Due to the method used for preparation of these beads, which will be explained in more detail below in view of the second aspect of the present invention, HCl that is formed as a side product is neutralized instantly (reaction performed under basic conditions). Therefore, no magnetite is lost and therefore the beads remain magnetic. This contributes to a more robust bead production. Furthermore, it could be shown that the diamine beads are less dependent on the choice of solvent in the sample preparation workflow. Whereas the classical FC beads function best with Acetonitrile as elution solvent for the analytes, the diamine beads are compatible with both Acetonitrile and Methanol.

### The Hypercrosslinked Magnetic Particle

Preferably, the hypercrosslinked magnetic particle has a particle size in the range of from 1 to 60 micrometer, as determined according to ISO 13320. More preferably, the particle size is in the range of from 5 to 55 micrometers, more preferably in the range of from 10 to 50 micrometers, more preferably in the range of from 15 to 45 micrometers, more preferably in the range of from 20 to 40 micrometers, and in particular in the range of from 20 to 35 micrometers.

As described above, the hypercrosslinked magnetic particle according to the invention comprises a polymer matrix (P) and at least one magnetic core (M). According to a preferred embodiment of the invention, the magnetic particle comprises more than one magnetic core (M), i.e. each particle preferably comprises at least one and, preferably, at least two magnetic cores (M). The magnetic core (M) comprises one or more magnetic nanoparticles, such as e.g. 1 to 20 magnetic nanoparticles, preferably 1 to 10, more preferably, 1 to 5 and most preferably 1 to 3 magnetic nanoparticles. Alternatively, it may comprise more than 20 nanoparticles and, preferably 100 to 1.5 million nanoparticles more preferably 750-750,000 nanoparticles, more preferably 1,750-320,000 nanoparticles, in particular 90,000-320,000 nanoparticles.

Preferably the amount of magnetic cores (M) is chosen so that a desired saturation magnetization saturation of the final particle is achieved. Preferably, the hypercrosslinked magnetic particle according to the invention has a saturation magnetization of at least 1 A m²/kg. Preferably, the saturation magnetization is at least 1 A m²/kg, more preferably at least 2 A m²/kg, more preferably at least 3 A m²/kg, more preferably at least 4 A m²/kg, more preferably at least 5 A m²/kg, more preferably at least 6 A m²/kg, more preferably at least 7 A m²/kg, more preferably at least 8 A m²/kg, more preferably at least 9 A m²/kg, and in particular at least 10 A m²/kg, such as in the range of from 10 A m²/kg to 20 A m²/kg, more preferably in the range of from 10 A m²/kg to 30 A m²/kg, as determined according to ASTM A 894/A 894M.

The hypercrosslinked magnetic particle of the present invention may, in principle, display any geometrical form, however, preferably, the particle is substantially spherical. As used herein, the term “substantially spherical” refers to particles with rounded shapes that are preferably non-faceted or substantially free of sharp corners. In certain embodiments, the substantially spherical particles typically have an average aspect ratio of less than 3:1 or 2:1, for example, an aspect ratio less than 1.5:1, or less than 1.2:1. In a certain embodiment, substantially spherical particles may have an aspect ratio of about 1:1. The aspect ratio (A_{R}) is defined as being a function of the largest diameter (dₘₐₓ) and the smallest diameter (dₘᵢₙ) orthogonal to it (A_{R}=dₘᵢₙ/dₘₐₓ). The diameters are determined via SEM or light microscope measurements.

The BET specific surface area of the hypercrosslinked magnetic particle as described above is preferably in the range of from 50 to 2500 m²/g, as determined according to ISO 9277. More preferably, the BET specific surface area of the magnetic particle is in the range of from 100 to 1500 m²/g and in particular in the range of from 300 to 1000 m²/g

According to a preferred embodiment of the present invention, the hypercrosslinked magnetic particle as described above is superparamagnetic. The term “superparamagnetic” is known to the person skilled in the art and refers to the magnetic property encountered in particular for particles smaller than a single magnetic mono-domain. Such particles steadily orient upon applying an external magnetic field until a maximum value of the global magnetization, dubbed saturation magnetization, is reached. They relax when removing the magnetic field, with no magnetic hysteresis (no remanence) at room temperature. In the absence of an external magnetic field, superparamagnetic particles exhibit a non-permanent magnetic moment due to thermal fluctuations of the dipole orientation (Neel relaxation) and particle position (Brownian relaxation).

In the hypercrosslinked magnetic particle, the at least one positive charge of the molecule comprising at least two nitrogen atoms within its structure is compensated by at least one corresponding anion. Preferably, the corresponding anion(s) are a carboxylate group of R² or are being selected from the group consisting of F⁻, Cl⁻, Br⁻, I⁻, At⁻ and OH⁻, preferably selected from the group consisting of Cl⁻, Br⁻, I⁻ and OH⁻ and is more preferably OH⁻.

The Molecule Comprising at Least Two Nitrogen Atoms within its Structure

According to a first preferred embodiment of the hypercrosslinked magnetic particle, the molecule comprising at least two nitrogen atoms within its structure has the general structure of formula Ia:

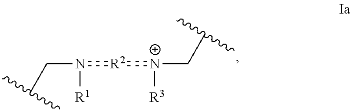

wherein the sinuous lines [symbol] represent the crosslinked polymer; R¹, R³ and R² together with the nitrogen atoms form an aromatic ring system comprising 3, 5, 7 or 9 carbon atoms; wherein the bonds connecting R² with each nitrogen atom are aromatic bonds; and wherein the molecule has a positive charge, which is compensated by a corresponding anion, preferably OH⁻. Preferably, the molecule comprising at least two nitrogen atoms within its structure having the general structure of formula Ia R² comprises one carbon atom and R¹, R³ together comprise 2, 4, 6 or 8 carbon atoms, wherein the bonds connecting R² with each nitrogen atom are aromatic bonds; and wherein the molecule has a positive charge, which is compensated by a corresponding anion, preferably OH⁻. More preferably, the molecule comprising at least two nitrogen atoms within its structure has the structure Ia-1:

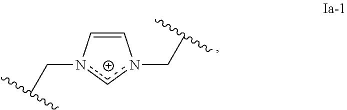

wherein the sinuous lines [symbol] represent the crosslinked polymer; and wherein the positive charge is compensated by a corresponding anion, which is preferably selected from the group described above, more preferably OH⁻.

According to another preferred embodiment of the hypercrosslinked magnetic particle, the molecule comprising at least two nitrogen atoms within its structure has the general structure of formula Ib:

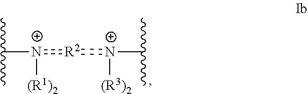

wherein
[symbol] the sinuous lines represent the crosslinked polymer;
R¹, R³ are independently selected from the group consisting of C1-C10-alkyl, C1-C10-alkenyl, and —(—O—CH₂—CH₂—)ₙ—O—CH₃ with n being an integer in the range of from 1 to 15, wherein each of R¹, R³ may have at least one further substituent selected from the group consisting of hydrogen, C1-C5-alkyl, C5-C12-aryl, C4-C10-heteroaryl, and wherein R¹ and R³ are separate;
R² is selected from the group consisting of C1-C10-alkyl, C1-C10-alkenyl, and —(—O—CH₂—CH₂—)ₙ—O— with n being an integer in the range of from 1 to 15;
   wherein the bonds connecting R² with each nitrogen atom are single bonds; and wherein the molecule has two positive charges, which are compensated by corresponding anions, preferably selected from the group described above, more preferably OH⁻.

Preferably, in the molecule comprising at least two nitrogen atoms within its structure having the general structure of formula Ib:
R¹, R³ are independently selected from the group consisting of C1-C10-alkyl, preferably from the group consisting of C1-C5-akyl;
R² is selected from the group consisting of C1-C10-alkyl, preferably from the group consisting of C2-C8-akyl;
   wherein the bonds connecting R² with each nitrogen atom are single bonds; and wherein the molecule has two positive charges, which are compensated by corresponding anions, preferably selected from the group described above, more preferably OH⁻.

More preferably, the molecule comprising at least two nitrogen atoms within its structure has the structure Ib-1:

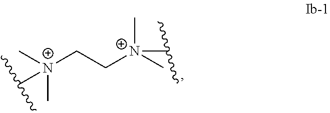 [SMILES: *[N+](C)(C)CC[N+](*)(C)C]

wherein the sinuous lines [symbol] represent the crosslinked polymer; and wherein the positive charges are compensated by corresponding anions, preferably selected from the group described above, more preferably OH⁻.

According to another preferred embodiment of the hypercrosslinked magnetic particle, the molecule comprising at least two nitrogen atoms within its structure has the general structure of formula Ic:

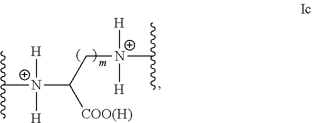

wherein
[symbol] the sinuous lines represent the crosslinked polymer; and
m is an integer in the range of from 1 to 10, preferably in the range of from 2 to 8, more preferred in the range of from 3 to 6;
   wherein COO(H) represents a carboxyl(ate) group; and the molecule has two positive charges which are compensated by corresponding anions, preferably OH⁻.

Preferably, the molecule comprising at least two nitrogen atoms within its structure having the general structure of formula Ic has the structure Ic-1:

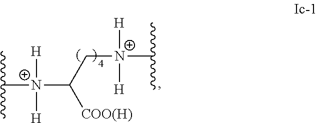

wherein the sinuous lines [symbol] represent the crosslinked polymer; and wherein the positive charges are compensated by corresponding anions, preferably OH⁻.

According to another preferred embodiment of the hypercrosslinked magnetic particle, the molecule comprising at least two nitrogen atoms within its structure has the general structure of formula Id:

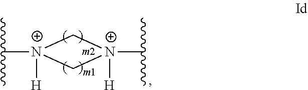

wherein the sinuous lines [symbol] represent the crosslinked polymer; m1 and m2 are independently integers in the range of from 2 to 10, preferably in the range of from 2 to 5; and wherein the molecule has two positive charges which are compensated by corresponding anions, preferably OH⁻. Preferably, the molecule comprising at least two nitrogen atoms within its structure has the general structure of formula Id-1:

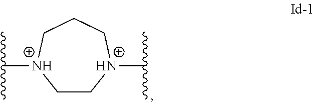 [SMILES: *[NH+]1CCC[NH+](*)CC1]

wherein the sinuous lines [symbol] represent the crosslinked polymer; and wherein the two positive charges are compensated by corresponding anions, preferably OH⁻.

### The Magnetic Core (M)

As described above, the magnetic particles according to the invention comprise at least one magnetic core (M) and preferably at least two magnetic cores (M). Preferably, the at least one magnetic core (M) comprises a compound selected from the group consisting of metal, metal carbide, metal nitride, metal sulfide, metal phosphide, metal oxide, metal chelate and a mixture of two or more thereof. The at least one magnetic core (M) may also comprise an alloy with a metal such as gold, silver, platinum or copper.

It is to be understood that each magnetic core (M) may comprise a mixture of two or more of the above-mentioned group, i.e. two or more of a metal, metal carbide, metal nitride, metal sulfide, metal phosphide, metal oxide, a metal chelate and a mixture of two or more thereof. Further, mixtures of two or more different metals, two or more different metal oxides, two or more different metal carbides, two or more different metal nitrides, two or more different metal sulphides, two or more different metal phosphides, two or more different metal chelates are conceivable.

Further, it is to be understood that in case the magnetic particle according to the invention comprises more than one magnetic core (M), each of the magnetic cores (M) present in a single particle may be the same or may differ from each other. Preferably, all magnetic cores (M) comprised in one magnetic particle are the same.

More preferably, the at least one magnetic core (M) comprises a metal oxide or a metal carbide.

In a preferred embodiment, the at least one magnetic core (M) comprises a metal, metal carbide, metal nitride, metal sulfide, metal phosphide, metal oxide, or metal chelate comprising at least one transition metal. Preferred transition metals according to the invention include, but are not limited to, chromium, manganese, iron, cobalt, nickel, zinc, cadmium, nickel, gadolinium, copper, and molybdenum. More preferably, the metal, metal carbide, metal nitride, metal sulfide, metal phosphide, metal oxide, or metal chelate comprises at least iron. More preferably, the at least one magnetic core (M) comprises a metal oxide or a metal carbide, more preferably, an iron oxide, in particular an iron oxide selected from the group consisting of Fe₃O₄, α-Fe₂O₃, γ-Fe₂O₃, MnFeₚO_{q}, CoFeₚO_{q}, NiFeₚO_{q}, CuFeₚO_{q}, ZnFeₚO_{q}, CdFeₚO_{q}, BaFeₚO and SrFeₚO, wherein p and q vary depending on the method of synthesis, and wherein p is preferably an integer of from 1 to 3, more preferably 2, and wherein q is preferably 3 or 4, most preferably Fe₃O₄.

Thus, the present invention also relates to a hypercrosslinked magnetic particle as described above, wherein the at least one magnetic core (M) comprises at least one magnetic nanoparticle, preferably at least one iron oxide nanoparticle, more preferably a Fe₃O₄-nanoparticle.

The magnetic core (M) preferably comprises, more preferably consists of nanoparticles and a coating C1.

### Nanoparticles

The Nanoparticles are preferably the part which displays the magnetism, preferably superparamagnetism of a particle. Nanoparticles are sometimes also referred to as “magnetic nanoparticles” herein.

Preferably, the at least one nanoparticle comprises, preferably consists of, at least one magnetic, preferably superparamagnetic, nanoparticle and optionally one coating, such as a coating C2.

As used herein, the term “nanoparticle” refers to a particle being less than 100 nanometers in at least one dimension, i.e. having a diameter of less than 100 nm. Preferably, the nanoparticle according to the invention has a diameter in the range of from 1 to 20 nm, preferably 4 to 15 nm, as determined according to TEM-measurements. Thus, according to a preferred embodiment, the present invention also relates to a magnetic particle as described above, as well as to a magnetic particle obtained or obtainable by the above-described method, wherein the magnetic particle comprises at least one magnetic core (M) which comprises at least one nanoparticle and optionally one coating, such as a coating C2.

Each nanoparticle(s), preferably has/have a diameter in the range of from 1 to 20 nm, preferably 4 to 15 nm, as determined according to TEM-measurements. Preferably, the at least one magnetic nanoparticle is superparamagnetic.

The magnetic core (M) may comprise only one nanoparticle or more than one nanoparticle. In one embodiment, it comprises from 1 to 20 nanoparticles. In another embodiment, it comprises 100 to 1.5 million nanoparticles more preferably 750-750,000 nanoparticles, more preferably 1,750-320,000 nanoparticles, in particular 90,000-320,000 nanoparticles. The nanoparticles may be present as magnetic core in the form of individual (i.e. separate) particles or they may for aggregates consisting of several nanoparticles. Theses aggregates may have different sizes depending on the number of included nanoparticles. Typically, so called supraparticles are formed, which are described further below in more detail. In the case of a magnetic core comprising 100 or more nanoparticles, the nanoparticles typically form such supraparticles.

### A Magnetic Core (M) Comprising 1-20 Nanoparticles

According to a first embodiment, the magnetic core (M) comprises, preferably consists of, 1-20 magnetic nanoparticles and optionally a coating C2, i.e. one magnetic nanoparticle, optionally with the coating C2, forms the nanoparticle of the magnetic core (M). Typically, the magnetic core comprises 1 to 20 magnetic nanoparticles, preferably 1 to 10, more preferably, 1 to 5 and most preferably 1 to 3 nanoparticles.

Preferably, in this case, the nanoparticle, comprises, more preferably consists of a compound selected from the group consisting of metal, metal carbide, metal nitride, metal sulfide, metal phosphide, metal oxide, metal chelate and a mixture of two or more thereof. It is to be understood that each nanoparticle may comprise, preferably consist of, a mixture of two or more of the above mentioned group, i.e. two or more of a metal, metal carbide, metal nitride, metal sulfide, metal phosphide, metal oxide, metal chelate and a mixture of two or more thereof. Further, mixtures of two or more different metals, two or more different metal oxides, two or more different metal carbides, two or more different metal nitrides, two or more different metal sulphides, two or more different metal chelates or two or more different metal phosphides are conceivable. More preferably, the nanoparticle comprises, more preferably consists of a metal oxide or a metal carbide. In a preferred embodiment, the metal is a transition metal. Preferred transition metals according to the invention include, but are not limited to, chromium, manganese, iron, cobalt, nickel, zinc, cadmium, nickel, gadolinium, copper, and molybdenum. Most preferably, the metal is iron.

Thus, according to a particularly preferred embodiment, the nanoparticle comprises, more preferably consists of a metal oxide, most preferably iron oxide, in particular Fe₃O₄.

According to this embodiment, it is preferred that in case more than one magnetic cores (M) are present in the magnetic particle, these magnetic cores (M) are not aggregated with each other. Preferably, these particles are substantially evenly distributed within the polymer matrix.

### A Magnetic Core (M) Comprising a Supraparticle

According to a another preferred embodiment, the magnetic core (M) comprises more than 20 nanoparticles, and, typically more than 100 nanoparticles, wherein these nanoparticles are preferably aggregated with each other to form a supraparticle. More preferably, in this case, the magnetic core (M) comprises a supraparticle consisting of aggregated, coated, nanoparticles. Preferably, in this case, the magnetic core (M) comprises a supraparticle which comprises between 100 to 1.5 million nanoparticles more preferably 750-750,000 nanoparticles, more preferably 1,750-320,000 nanoparticles, in particular 90,000-320,000 nanoparticles. Preferably, each nanoparticle is coated with at least one coating C2. Preferably in this case, the magnetic core (M) thus comprises, preferably consists of, the supraparticle, which consist of, coated, nanoparticles being aggregated with each other, wherein the nanoparticles are coated with at least one coating C2, and wherein the coating is preferably deposited on the surface of the nanoparticles. The supraparticle may preferably also be coated with a coating C1.

Thus according to this another preferred embodiment of the invention, the magnetic particle according to the invention comprises more than 20 magnetic nanoparticles, and preferably 100 to 1.5 million nanoparticles, wherein said nanoparticles form at least one supraparticle. Each of the nanoparticles in the supraparticle is typically coated with at least one coating C2 and the supraparticle is typically coated with at least one coating C1.

Preferably, the coating C2 is a coating which covers at least a part, preferably the whole surface, of each nanoparticle. Preferably, also in this case, each nanoparticle comprises, more preferably consists of, a compound selected from the group consisting of metal, metal carbide, metal nitride, metal sulfide, metal phosphide, metal oxide, metal chelate and a mixture of two or more thereof.

It is to be understood that each nanoparticle present in the supraparticle may comprise, preferably consist of, a mixture of two or more of the above-mentioned group, i.e. two or more of a metal, metal carbide, metal nitride, metal sulfide, metal phosphide, metal oxide, metal chelate and a mixture of two or more thereof. Further, mixtures of two or more different metals, two or more different metal oxides, two or more different metal carbides, two or more different metal nitrides, two or more different metal sulphides, two or more different metal chelates or two or more different metal phosphides are conceivable. More preferably, each nanoparticle in the supraparticle comprises, more preferably consists of, a metal oxide or a metal carbide. In a preferred embodiment, the metal is a transition metal. Preferred transition metals according to the invention include, but are not limited to, chromium, manganese, iron, cobalt, nickel, zinc, cadmium, nickel, gadolinium, copper, and molybdenum. Most preferably, the metal is iron. According to a particularly preferred embodiment, each nanoparticle comprised in the supraparticle is a metal oxide nanoparticle, most preferably an iron oxide nanoparticle, in particular a Fe₃O₄-nanoparticle.

Thus, the present invention also relates to a magnetic particle as described above, as well as to a magnetic particle obtained or obtainable by the above described method, wherein the magnetic core (M) comprises or preferably consists of a supraparticle consisting of aggregated at least 20 magnetic nanoparticles wherein the nanoparticles are preferably being coated with at least one coating C2.

Preferably, the magnetic core (M) including the optional, at least one coating C1 has a diameter in the range of from 80 to 500 nm, more preferably 150 to 400 nm, and most preferably 200 to 300 nm, as determined according to DLS (ISO 22412).

The coating C2

As coating C2, in general any coating known to those skilled in the art is conceivable. Preferably, the coating C2 is, however, selected from at least one member of the group consisting of dicarboxylic acids, tricarboxylic acids, polyacrylic acid, amino acids, surfactants and fatty acids. It is to thus be understood that the aforementioned group includes salts and derivatives, such as esters and polymers, of the mentioned compounds. Thus, the coating C2 is preferably selected from at least one member of the group consisting of dicarboxylic acids, dicarboxylic acid salts, dicarboxylic acid derivatives, tricarboxylic acids, tricarboxylic acid salts, tricarboxylic derivatives, polyacrylic acid, polyacrylic acid salts, polyacrylic acid derivatives, amino acids, amino acid salts, amino acid derivatives, surfactants, salt of surfactants, fatty acids, fatty acid salts and fatty acid derivatives.

As used herein, the terms coated or coating are used to refer to the process of adsorption, van der Waals and/or non-polar group interactions (e.g., chemisorption or physical adsorption), or covalent binding of the magnetic nanoparticle or supraparticle core and the coating C2 or C1 or between two or more coatings, if present.

Preferably as fatty acids, fatty acid salts or fatty acid derivatives, such compounds are chosen which are capable of binding to the surface of the supraparticle, thereby preferably stabilizing the supraparticle. A fatty acid employed as coating C2 is preferably a single chain of alkyl groups containing from 8 to 22 carbon atoms with a terminal carboxyl group (—COOH) and high affinity adsorption (e.g., chemisorption or physical adsorption) to the surface of the magnetic particle. The fatty acid has multiple functions including protecting the magnetic particle core from oxidation and/or hydrolysis in the presence of water, which can significantly reduce the magnetization of the nanoparticle (Hutten, et al. (2004) J. Biotech. 112:47-63); stabilizing the nanoparticle core; and the like. The term “fatty acid” includes saturated or unsaturated, and in particular unsaturated fatty acids. Exemplary saturated fatty acids include lauric acid, myristic acid, palmitic acid, stearic acid, arachidic acid, propionic acid, butyric acid, valeric acid, caproic acid, enanthic acid, caprylic acid, pelargonic acid, capric acid, undecylic acid, tridecylic acid, pentadecylic acid, margaric acid, nonadecylic acid, heneicosylic acid, behenic acid, tricosylic acid, lignoceric acid, pentacosylic acid, cerotic acid, heptacosylic acid, montanic acid, nonacosylic acid, melissic acid, henatriacontylic acid, lacceroic acid, psyllic acid, geddic acid, ceroplastic acid, hexatriacontylic acid, heptatriacontanoic acid and octatriacontanoic acid and the like. Exemplary unsaturated fatty acids include oleic acid, linoleic acid, linolenic acid, arachidonic acid, hexadecatrienoic acid, stearidonic acid, eicosatrienoic acid, eicosatetraenoic acid, eicosapentaenoic acid, heneicosapentaenoic acid, docosapentaenoic acid, clupanodonic acid, docosahexaenoic acid, tetracosapentaenoic acid, tetracosahexaenoic acid, calendic acid, eicosadienoic acid, docosadienoic acid, adrenic acid, docosapentaenoic acid, tetracosatetraenoic acid, tetracosapentaenoic acid, 5-dodecenoic acid, 7-tetradecenoic acid, palmitoleic acid, vaccenic acid, paullinic acid, 15-docosenoic acid, 17-tetracosenoic acid, elaidic acid, gondoic acid, mead acid, erucic acid, nervonic acid, rumenic acid, calendic acid, jacaric acid, eleostearic acid, catalpic acid, punicic acid, rumelenic acid, parinaric acid, bosseopentaenoic acid, pinolenic acid, podocarpic acid and the like. The fatty acid can be synthetic or isolated from a natural source using established methods. Moreover, a fatty acid can be a derivative such as a fatty acid enol ester (i.e., a fatty acid reacted with the enolic form of acetone), a fatty ester (i.e., a fatty acid with the active hydrogen replaced by the alkyl group of a monohydric alcohol), a fatty amine or fatty amide, or in particular embodiments, a fatty alcohol as described above. A particularly preferred fatty acid is oleic acid.

A surfactant, as used in the context of the instant invention, is an organic compound that is amphipathic, i.e., containing both hydrophobic groups and hydrophilic groups. Preferably surfactants are chosen which are capable of binding to the surface of the supraparticle thereby preferably stabilizing the supraparticle surfactants with a variety of chain lengths, hydrophilic-lipophilic balance (HLB) values and surfaces charges can be employed depending upon the application. Preferably, the surfactant according to the invention is a quateranary ammonium salt, alkylbenzenesulfonates, lignin sulfonates, polyoxylethoxylate, or sulfate ester. Non-limiting examples of surfactants are cetyltrimethylammonium bromide, cetyltrimethylammonium chloride, nonyphenolpolyethoxylates (i.e. NP-4, NP-40 and NP-7), sodium dodecylbenzenesulfonate, ammonium lauryl sulfate, sodium laureth sulfate, sodium myreth sulfate, docusate, perfluorooctanesulfonate, perfluorobutanesulfonate, alkyl-aryl ether phosphates, alkyl ether phosphates, sodium stearate, 2-Acrylamido-2-methylpropane sulfonic acid, ammonium perfluorononanoate, magnesium laureth sulfate, perfluorononanoic acid, perfluorooctanoic acid, phospholipids, potassium lauryl sulfate, sodium alkyl sulfate, sodium dodecyl sulfate, sodium laurate, sodium lauroyl sarcosinate, sodium nonanoyloxybenzenesulfonate, sodium pareth sulfate, behentrimonium chloride, benzalkonium chloride, benzethonium chloride, bronidox, dimethyldioctadecylammonium bromide, dimethyldioctadecylammonium chloride, lauryl methyl gluceth-10 hydroxypropyl dimonium chloride, octenidine dihydrochloride, olaflur, N-oleyl-1,3-propanediamine, stearalkonium chloride, tetramethylammonium hydroxide, thonzonium bromide, cetomacrogol 1000, cetostearyl alcohol, cetyl alcohol, cocamide DEA, cocamide MEA, decyl polyglucose, disodium cocoamphodiacetate, glycerol monostearate, polyethylene glycol isocetyl ether, octylphenoxypolyethoxyethanol, lauryl glucoside, maltosides, monolaurin, mycosubtilin, nonoxynols, octaethylene glycol monododecyl ether, N-octyl beta-D-thioglucopyranoside, octyl glucoside, oleyl alcohol, pentaethylene glycol monododecyl ether, polidocanol, poloxamer, polyethoxylated tallow amine, polyglycerol polyricinoleate, polysorbate, sorbitan, sorbitan monolaurate, sorbitan monostearate, sorbitan tristearate, stearyl alcohol, surfactin, Triton X-100, Tween 80, cocamidopropyl betaine, cocamidopropyl hydroxysultaine, dipalmitoylphosphatidylcholine, hydroxysultaine, lLauryldimethylamine oxide, lecithin, myristamine oxide, peptitergents, sodium lauroamphoacetate and bis(2-ethylhexyl)sulfosuccinic ester.

The term “amino acids” as used within the meaning of the present invention refers to natural or unnatural amino acids or amino acid derivatives as well as to salts of amino acids. Preferably, amino acids are chosen which are capable of binding to the surface of the supraparticle thereby preferably stabilizing the supraparticle. Exemplary amino acids include cysteine, methionine, histidine, alanine, arginine, asparagine, aspartic acid, glutamic acid, glutamine, glycine, isoleucine, leucine, lysine, phenylalanine, proline, serine, threonine, tryptophan, tyrosine, valine, selenocysteine, pyrrolysine, cysteine, dehydroalanine, enduracididine, lanthionine, norvaline and derivatives thereof.

The term “dicarboxylic acid” within the meaning of the present invention refers to a hydrocarbon or substituted hydrocarbon containing two carboxylic acid functional groups (i.e., R¹—(C(O)OH)₂), where R¹ is (a) a linear hydrocarbon containing from 0-18 carbon units or (b) a cyclic hydrocarbon containing 3-8 carbon units, either as aromatic or non-aromatic rings. The term includes salts and derivatives of fatty acids, such as esters of fatty acids. Representative dicarboxylic acids are e.g. propanedioic acid, butanedioic acid, pentanedioic acid, hexanedioic acid, heptanedioic acid, octanedioic acid, nonanedioic acid, decanedioic acid, undecanedioic acid, dodecanedioic acid, hexadecanedioic acid, maleic acid, fumaric acid, glutaconic acid, traumatic acid, muconic acid, glutinic acid, citraconic acid, mesaconic acid, malic acid, aspartic acid, glutamic acid, tartronic acid, tartaric acid, diaminopimelic acid, saccharic acid, mesoxalic acid, oxaloacetic acid, acetonedicarboxylic acid, arabinaric acid, phthalic acid, isophthalic acid, terephthalic acid, diphenic acid, 2,6-naphthalenedicarboxylic acid.

The term “tricarboxylic acid” within the meaning of the present invention refers to a hydrocarbon or substituted hydrocarbon containing three carboxylic acid functional groups (i.e., R¹—(C(O)OH)₃), where R¹ is (a) a linear hydrocarbon containing from 3-18 carbon units or (b) a cyclic hydrocarbon containing 3-8 carbon units, either as aromatic or non-aromatic rings. The term includes salts and derivatives of fatty acids, such as esters of fatty acids. Representative tricarboxylic acids are e.g. citric acid (2-hydroxypropane-1,2,3 tricarboxylic acid), isocitric acid (1-hydroxypropane-1,2,3 tricarboxylic acid), aconitic acid (prop-1-ene-1,2,3 tricarboxylic acid), propane-1,2,3-tricarboxylic acid, trimellitic acid (benzene-1,2,4-tricarboxylic acid), trimesic acid (benzene-1,3,5-tricarboxylic acid), oxalosuccinic acid (1-oxopropane-1,2,3-tricarboxylic acid) or hemimellitic acid (benzene-1,2,3-tricarboxylic acid). Preferably, the tricarboxylic acid is citric acid including citrates, i.e. salts and derivatives of citric acid.

Preferably, C2 is selected from the group consisting of citric acid, histidine, CTAB, CTAC, sodium oleate, polyacrylic acid or mixtures of two or more thereof (including the respective salts or derivatives thereof). Thus, the present invention also relates to a magnetic particle as described above, as well as to a magnetic particle obtained or obtainable by the above-described method, wherein the magnetic core (M) preferably consists of, a supraparticle consisting of aggregated magnetic nanoparticles with at least one coating C2, wherein the at least one coating C2 is selected from the group consisting of citrate, histidine, CTAB, CTAC, sodium oleate, polyacrylic acid or mixtures of two or more thereof.

Preferably the amount of coating C2 is in the range of from 1 to 80% by weight, more preferably in the range of from 5 to 70% by weight, more preferably in the range of from 10 to 50% by weight, most preferably 20 to 40% based on the total weight of the sum of C2 and the supraparticle.

### The Coating C1

As described above, the magnetic core (M), preferably comprises, more preferably consists of, magnetic nanoparticles and a coating C1. Thus, the present invention also relates to a magnetic particle as described above, as well as to a magnetic particle obtained or obtainable by the above-described method, wherein the at least one magnetic core (M) further comprises a coating C1.

The coating C1 is preferably deposited on the surface of the magnetic core (M). It is to be understood that between coating C1 and the magnetic core (M), further separating layers may exist, however, according to a preferred embodiment, C1 is coated directly on the magnetic core (M).

Preferably, the coating C1 surrounds the whole surface of the magnetic core (M).

In principle, any suitable coating known to those skilled in the art may be employed. Preferably, the coating C1 is selected from the group consisting of tensides, silica, silicates, silanes, phosphates, phosphonates, phosphonic acids and mixtures of two or more thereof.

Thus, the present invention also relates to a magnetic particle as described above, as well as to a magnetic particle obtained or obtainable by the above-described method, comprising at least one magnetic core (M), wherein the at least one magnetic core (M) comprises at least one coating C1, and wherein the coating C1 is selected from the group consisting of tensides, silica, silicates, silanes, phosphates, phosphonates, phosphonic acids and mixtures of two or more thereof, preferably the coating is a tenside coating.

Preferably, the coating C1 is selected from the group consisting of silica, tetraethyl orthosilicate, 3-(trimethoxysilyl)propyl methacrylate, vinyltrimethoxysilane, vinyltriethoxysilane, allyltrimethoxysilane, allyltriethoxysilane, triethoxyvinylsilane, 3-(trimethoxysilyl)propyl acrylate, trimethoxy(7-octen-1-yl)silane, trimethoxymethylsilane, triethoxymethylsilane, ethyltrimethoxysilane, triethoxy(ethyl)silane, trimethoxyphenylsilane, trimethoxy(2-phenylethyl)silane trimethoxy(propyl)silane, n-propyltriethoxysilane, isobutyl(trimethoxy)silane, isobutyltriethoxysilane, vinylphosphonic acid, dimethyl vinylphosphonate, diethyl vinylphosphonate, diethyl allylphosphonate, diethyl allyl phosphate, diethyl (2-methylallyl)phosphonate, octylphosphonic acid, butylphosphonic acid, decylphosphonic acid, hexylphosphonic acid, hexadecylphosphonic acid, n-dodecylphosphonic acid, lauric acid, myristic acid, palmitic acid, stearic acid, and arachidic acid, propionic acid, butyric acid, valeric acid, caproic acid, enanthic acid, caprylic acid, pelargonic acid, capric acid, undecylic acid, tridecylic acid, pentadecylic acid, margaric acid, nonadecylic acid, heneicosylic acid, behenic acid, tricosylic acid, lignoceric acid, pentacosylic acid, cerotic acid, heptacosylic acid, montanic acid, nonacosylic acid, melissic acid, henatriacontylic acid, lacceroic acid, psyllic acid, geddic acid, ceroplastic acid, hexatriacontylic acid, heptatriacontanoic acid, octatriacontanoic acid, oleic acid, linoleic acid, linolenic acid, arachidonic acid, hexadecatrienoic acid, stearidonic acid, eicosatrienoic acid, eicosatetraenoic acid, eicosapentaenoic acid, heneicosapentaenoic acid, docosapentaenoic acid, clupanodonic acid, docosahexaenoic acid, tetracosapentaenoic acid, tetracosahexaenoic acid, calendic acid, eicosadienoic acid, docosadienoic acid, adrenic acid, docosapentaenoic acid, tetraco-satetraenoic acid, tetracosapentaenoic acid, 5-dodecenoic acid, 7-tetradecenoic acid, palmitoleic acid, vaccenic acid, paullinic acid, 15-docosenoic acid, 17-tetracosenoic acid, elaidic acid, gondoic acid, mead acid, erucic acid, nervonic acid, rumenic acid, calendic acid, jacaric acid, eleostearic acid, catalpic acid, punicic acid, rumelenic acid, parinaric acid, bosseopentaenoic acid, pinolenic acid, podocarpic acid and mixtures of two or more thereof.

Preferably, each magnetic core (M) comprises the coating C1 in an amount of from 1 to 40% by weight, preferably from 2 to 15% by weight, more preferably from 5 to 10% by weight, based on the total weight of at least one magnetic core (M).

According to one preferred embodiment of the invention, the coating C1 comprises vinyl or acryl groups.

### The Polymer Matrix (P)

As described above, each particle comprises besides the at least one magnetic core (M) a polymer matrix (P).

Preferably, the polymer matrix (P) is a porous polymer matrix, preferably a porous polymer matrix comprising pores having a pore size smaller than 100 nm, more preferably smaller than 100 nm, more preferably smaller than 90 nm, more preferably smaller than 80 nm, more preferably smaller than 70 nm, more preferably smaller than 60 nm, more preferably smaller or equal to 50 nm, such as in the range of from 0.5 nm to 50 nm, preferably in the range of from 1 to 20 nm as determined according to ISO 15901.

Thus, the present invention also relates to a magnetic particle as described above, as well as to a magnetic particle obtained or obtainable by the above-described method, wherein the polymer matrix (P) is a porous polymer matrix comprising pores having a pore size smaller than 100 nm, preferably smaller or equal to 50 nm, as determined according to ISO 15901.

Preferably at least 90% of all pores present in the polymer matrix have a pore size smaller than 10 nm and at least 50% of all pores present in the polymer matrix have a pore size smaller than 5 nm, as determined according to ISO 15901.

According to a particularly preferred embodiment, the polymer matrix does not comprise macropores, i.e. pores having a pore size larger than 50 nm.

Preferably, the particle comprises the polymer matrix (P) in an amount in the range of from 40 to 98% by weight, more preferably in the range of from 50 to 95% by weight, more preferably in the range of from 60 to 90% by weight, and most preferably in the range of from 70 to 85% by weight, based on the total weight of the particle.

The polymer matrix (P) comprises a crosslinked polymer, wherein the polymer preferably comprises a co-polymer obtained or obtainable by a method comprising a polymerization of at least two different monomeric building blocks selected from the group consisting of styrene, functionalized styrenes, vinylbenzylchloride, divinylbenzene, vinylacetate, methylmethaacrylate and acrylic acid.

At least one monomeric building block used has functional groups reactive towards amine groups or amine groups. Preferably, the functional group reactive towards amine groups is selected from the group of halogenated C1-C3-alkyl group, halogen atom, epoxy group and activated carboxy group, preferably -acid halide or acid anhydride or succinimide. According to a preferred embodiment, the functional group reactive towards amine groups is a halogenated C1-C3-alkyl group, more preferably a —CH₂—Cl group. According to a preferred embodiment vinylbenzyl chloride is employed as monomeric building block having functional groups reactive towards amine groups.

Further, at least one monomeric building block is a crosslinking agent, thus an agent with which in the resulting polymer a crosslinking is achieved. Suitable agents for crosslinking polymers are known to those skilled in the art, and include, but are not limited to building block such as divinylbenzene, bis(vinylphenyl)ethane, bis(vinylbenzyloxy)hexane, bis(vinylbenzyloxy)dodecane and derivatives of those.

Thus, the polymer matrix comprises a crosslinked polymer, this crosslinked polymer being obtained or obtainable by a method comprising co-polymerizing suitable monomeric building blocks in the presence of at least one monomeric building block which has functional groups reactive towards amine groups or amine groups and at least one monomeric building block which is a crosslinking agent.

Preferably, the co-polymer obtained or obtainable by a method comprising a polymerization of at least two different monomeric building blocks selected from the group consisting of the following monomers:

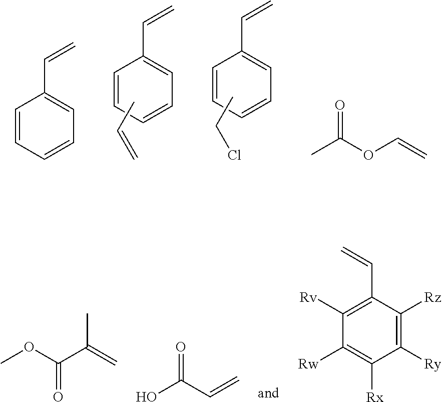

with R^{v}, R^{w}, R^{x}, R^{y} and R^{z}, are, independently of each other selected from the group consisting of —N₃, —NH₂, —Br, —I, —F, —NR′R″, —NR′R″R′″, —COOH, —CN, —OH, —OR′, —COOR′, —NO₂, —SH₂, —SO₂, —R′(OH)ₓ, —R′(COOH)ₓ, —R′(COOR″)ₓ, —R′(OR″)ₓ, —R′(NH2)ₓ, —R′(NHR″)ₓ, —R′(NR″R′″)ₓ, —R′(Cl)ₓ, —R′(I)ₓ, —R′(Br)ₓ, —R′(F)ₓ, R′(CN)ₓ, —R′(N₃)ₓ, —R′(NO₂)ₓ, —R′(SH₂)ₓ, —R′(SO₂)ₓ, alkyl, aryl, cycloalkyl, heteroaryl, heterocycloalkyl and with R′, R″ and R′″ being, independently of each other, selected from the group consisting of alkyl, aryl, cycloalkyl, heteroaryl, heterocycloalkyl, halides, hydrogen, sulfides, nitrates and amines, and wherein x is an integer in the range of from 1 to 3.

According to a preferred embodiment divinylbenzene is employed as crosslinking agent.

According to a further preferred embodiment, divinylbenzene is employed as crosslinking agent and vinylbenzyl chloride is employed as monomeric building block having functional groups reactive towards amine groups.

Preferably, the polymer matrix is obtained or obtainable by a method comprising co-polymerizing monomeric building blocks, wherein 5-90 vol % of all monomeric building blocks are crosslinking agents. Preferably, in the resulting polymer a crosslinking degree of at least 5% is obtained.

### Hypercrosslinking

According to the invention, the polymer matrix (P) comprises a crosslinked co-polymer obtained or obtainable by a method comprising the polymerization of at least two different monomeric building blocks as described above, whereby a crosslinked polymer is obtained, wherein the crosslinked polymer is further hypercrosslinked. Thus, the polymer matrix comprises, in particular consists of a hypercrosslinked polymer.

The term “hypercrosslinked” as used herein refers to a type of multiple crosslinking resulting in a rigid three-dimensional network. The hypercrosslinking is achieved by subjecting the crosslinked polymer to a chemical reaction, thereby obtaining the hypercrosslinked polymer. According to the invention, the polymer matrix comprises, preferably consists of, at least one crosslinked polymer, which comprises at least two functional groups reactive towards amine groups or at least two amine groups; said groups react in a chemical reaction with a molecule comprising at least two amine groups within its structure or a molecule comprising at least two functional groups reactive towards amine groups thereby forming at least one hypercrosslinking bond; and thereby obtaining a hypercrosslinked magnetic particle. Thus, the polymer matrix (P) is a polymer matrix being obtained or obtainable by further hypercrosslinking the crosslinked polymer with a molecule comprising at least two amine groups within its structure or a molecule comprising at least two functional groups reactive towards amine groups by a chemical reaction. As described above, the polymer matrix comprises at least one crosslinked polymer having at least one hypercrosslinking bond, wherein the hypercrosslinking bond comprises a molecule comprising at least two nitrogen atoms within its structure which are part of the hypercrosslinking bond; and having at least one positive charge.

The at least one magnetic core (M) is preferably embedded in the polymer matrix (P). The term “embedded” in this context is denoted to mean the magnetic core is preferably fully surrounded by the polymer matrix. Alternatively, it may be partially surrounded by the polymer matrix. In this case, the polymer matrix, however, immobilizes the magnetic core.

As described above, according to a preferred embodiment, the particle comprises at least two magnetic cores (M). In this case, it is to be understood, that each magnetic core (M) present in the particle is embedded in the polymer matrix (P). Thus, the present invention also relates to a magnetic particle as described above, wherein the at least two magnetic cores (M) are embedded in the polymer matrix (P).

### Method for Preparing the Hypercrosslinked Magnetic Particle

In a further aspect, the present invention also relates to a method of preparing a hypercrosslinked magnetic particle comprising a polymer matrix (P) and at least one magnetic core (M), wherein the polymer matrix comprises at least one crosslinked polymer having at least one hypercrosslinking bond, wherein the hypercrosslinking bond consists of a molecule comprising at least two nitrogen atoms within its structure which are part of the hypercros slinking bond, wherein the molecule comprising at least two nitrogen atoms within its structure has the general structure of formula I

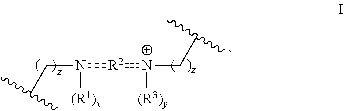

wherein
x, y are independently 1 or 2;
z is zero or 1;
R¹, R³ are independently selected from the group consisting of hydrogen, C1-C10-alkyl, C1-C10-alkenyl, C5-C10-cycloalkyl, C5-C12-aryl, C4-C10-heteroaryl and —(—O—CH₂—CH₂—)ₙ—O—CH₃ with n being an integer in the range of from 1 to 15, wherein each of R¹, R³ may have at least one further substituent selected from the group consisting of hydrogen, C1-C5-alkyl, C5-C12-aryl, C4-C10-heteroaryl, and wherein R¹ and R³ are separate or together with R² form an aliphatic or aromatic ring system;
R² is selected from the group consisting of C1-C10-alkyl, which is optionally substituted with a —COOH or COO⁻ group, C1-C10-alkenyl, C5-C10-cycloalkyl, C5-C12-aryl, C4-C10-heteroaryl and —(—O—CH₂—CH₂—)ₙ—O— with n being an integer in the range of from 1 to 15, wherein each cyclic structure having two or more ring systems has separated or annulated ring systems;
[symbol] the sinuous lines represent the crosslinked polymer;
   wherein the bonds connecting R² with each nitrogen atom are independently selected from the group consisting of single, double and aromatic bond; and
   wherein the molecule comprising at least two nitrogen atoms within its structure having the general structure of formula I has at least one positive charge;
   the method comprising:
(i) Providing a magnetic particle comprising a polymer matrix (P) and at least one magnetic core (M), wherein the polymer matrix (P) comprises, preferably consists of, at least one crosslinked polymer, which comprises at least two functional groups reactive towards amine groups;
(ii) providing a molecule comprising at least two amine groups within its structure;
(iii) reacting the groups reactive to amine groups of the magnetic particle provided in (i) with the amine groups of the molecule provided in (ii) thereby forming at least one hypercrosslinking bond; and thereby obtaining a hypercrosslinked magnetic particle.

One advantage of the inventive method is the avoidance of HCl as a side-product of the Friedel-Crafts reaction that is used for the hypercrosslinking in the “classical” approach. The HCl causes the magnetite inside the bead to dissolve, making the beads less magnetic. Further, the use of FeCl₃ as a catalyst can be avoided. This corrosive reagent is only very limited water soluble and in hydroxylized form tends to attach to storage/reaction vessels (e.g. glass or steel), making this an unpractical reagent to work with. In the end, the presence of at least one charge offers a functionality which is highly advantageous for analyte capturing, surprisingly independent whether the analyte has a charge of its own or not.

### Step (i)

In a preferred embodiment of the method, (i) comprises:
(i-1) providing at least one magnetic core (M);
(i-2) providing polymer precursor molecules comprising at least one kind of polymer precursor molecules having functional groups reactive towards amine groups or amine groups,
(i-3) polymerizing the polymer precursor molecules according to (i-2) in the presence of the at least one magnetic core (M), thereby forming a particle comprising the at least one magnetic core (M), embedded in a polymer matrix (P1), wherein the polymer matric (P1) preferably comprises, more preferably consists of, a crosslinked polymer having at least two functional groups reactive towards amine groups or at least two amine groups.
   Step (i-1)

As described above, the at least one magnetic core (M) provided according to (i-1) preferably comprises a compound selected from the group consisting of metal, metal carbide, metal nitride, metal sulfide, metal phosphide, metal oxide, metal chelate and a mixture of two or more thereof. The at least one magnetic core (M) may also comprise an alloy with a metal such as gold, silver, platinum, or copper. More preferably, the at least one magnetic core (M) comprises a metal oxide or a metal carbide, more preferably, the at least one magnetic core (M) comprises an iron oxide, in particular an iron oxide selected from the group consisting of Fe₃O₄, α-Fe₂O₃, γ-Fe₂O₃, MnFeₚO_{q}, CoFeₚO_{q}, NiFeₚO_{q}, CuFeₚO_{q}, ZnFeₚO_{q}—CdFeₚO_{q}, BaFeₚO and SrFeₚO, wherein p and q vary depending on the method of synthesis, and wherein p is preferably an integer of from 1 to 3, more preferably 2, and wherein q is preferably 3 or 4, and most preferably, the at least one magnetic core (M) comprises Fe₃O₄.

Thus, the present invention also relates to a method, as described above, and a magnetic particle obtained or obtainable by said method, wherein the at least one magnetic core (M comprises a metal oxide or a metal carbide, more preferably, the at least one magnetic core (M) comprises an iron oxide, in particular an iron oxide selected from the group consisting of Fe₃O₄, α-Fe₂O₃, γ-Fe₂O₃, MnFeₚO_{q}, CoFeₚO_{q}, NiFeₚO_{q}, CuFeₚO_{q}, ZnFeₚO_{q}—CdFeₚO_{q}, BaFeₚO and SrFeₚO, wherein p and q vary depending on the method of synthesis, and wherein p is preferably an integer of from 1 to 3, more preferably 2, and wherein q is preferably 3 or 4, and most preferably, the at least one magnetic core (M) comprises Fe₃O₄

As described above, the magnetic core (M) preferably comprises, more preferably consists of, magnetic nanoparticles and a coating C1.

According to a preferred embodiment, step (i-1) comprises:
(i-1.1) providing at least one magnetic nanoparticle, and
(i-1.2) coating the at least one nanoparticle with a coating C1, the coating C1 preferably being selected from a group consisting of tensides, silica, silicates, silanes, phosphates, phosphonates, phosphonic acids and mixtures of two or more thereof
   to give the magnetic core (M).
   Step (i-2)

Preferably, the polymer precursor molecules in (i-2) are selected from the group consisting of styrene, functionalized styrenes, vinylbenzylchloride, divinylbenzene, vinylacetate, methylmethaacrylate and acrylic acid.

At least one polymer precursor molecule used has functional groups reactive towards amine groups or amine groups. Preferably, the functional group reactive towards amine groups is selected from the group of halogenated C1-C3-alkyl group, halogen atom, epoxy group and activated carboxy group, preferably -acid halide or acid anhydride or succinimide. According to a preferred embodiment, the functional group reactive towards amine groups is a halogenated C1-C3-alkyl group, more preferably a —CH₂—Cl group. According to a preferred embodiment vinylbenzyl chloride is employed as polymer precursor molecule having functional groups reactive towards amine groups.

Further, at least one polymer precursor molecule is a crosslinking agent, thus an agent with which in the resulting polymer a crosslinking is achieved. Suitable agents for crosslinking polymers are known to those skilled in the art, and include, but are not limited to building block such as divinylbenzene, bis(vinylphenyl)ethane, bis(vinylbenzyloxy)hexane, bis(vinylbenzyloxy)dodecane and derivatives of those.

Thus, at least one polymer precursor molecule which has functional groups reactive towards amine groups or amine groups and at least one polymer precursor molecule which is a crosslinking agent are used.

Preferably, the at least two different polymer precursor molecules are selected from the group consisting of the following monomers:

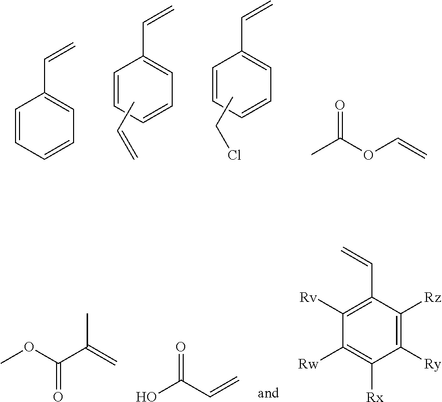

with R^{v}, R^{w}, R^{x}, R^{y} and R^{z}, are, independently of each other selected from the group consisting of —N₃, —NH₂, —Br, —I, —F, —NR′R″, —NR′R″R′″, —COOH, —CN, —OH, —OR′, —COOR′, —NO₂, —SH₂, —SO₂, —R′(OH)ₓ, —R′(COOH)ₓ, —R′(COOR″)ₓ, —R′(OR″)ₓ, —R′(NH₂)ₓ, —R′(NHR″)ₓ, —R′(NR″R′″)ₓ, —R′(Cl)ₓ, —R′(I)ₓ, —R′(Br)ₓ, —R′(F)ₓ, R′(CN)ₓ, —R′(N₃)ₓ, —R′(NO₂)ₓ, —R′(SH₂)ₓ, —R′(SO₂)ₓ, alkyl, aryl, cycloalkyl, heteroaryl, heterocycloalkyl and with R′, R″ and R′″ being, independently of each other, selected from the group consisting of alkyl, aryl, cycloalkyl, heteroaryl, heterocycloalkyl, halides, hydrogen, sulfides, nitrates and amines.

According to a preferred embodiment divinylbenzene is employed as crosslinking agent. According to a further preferred embodiment, divinylbenzene is employed as crosslinking agent and vinylbenzyl chloride is employed as polymer precursor molecule having functional groups reactive towards amine groups.

Preferably, the polymer matrix (P1) is obtained or obtainable by crosslinking a polymer with 5-90 vol % of a crosslinking agent, based on the total amount of the polymer.

Step (i-3)

In step (i-3), the polymer precursor molecules according to (i-2) are polymerized in the presence of the at least one magnetic core (M), thereby forming a particle comprising the at least one magnetic core (M), preferably the at least two magnetic cores (M), embedded in a polymer matrix (P1), wherein the polymer matrix (P1) preferably comprises, more preferably consists of, a crosslinked polymer, as described above and below. This crosslinked polymer matrix (P1) is then further hypercrosslinked in step (iii) to give the polymer matrix (P).

The polymerization in (i-3) is preferably a suspension polymerization. The term “suspension polymerization” refers to a system in which polymeric precursor molecules that are relatively insoluble in water are suspended as liquid droplets in an aqueous phase. Usually, a suspending agent is employed so as to maintain the suspension, and the resultant polymer is obtained as a dispersed solid phase. While the polymeric precursor molecules (aka monomeric building blocks) may be directly dispersed in a suspension polymerization system, hydrocarbon solvents or diluents are commonly employed with the monomers, such as n-heptane, isooctane, cyclohexane, benzene, toluene, and the like, including mixtures.

In the suspension polymerization system, a monomer mixture to be polymerized usually comprises the monomers, or, where desired, a polymer-in-monomer solution, the at least one magnetic core (M), solvent and, where employed, an initiator.

The polymerization in (i-3) is preferably carried out in the presence of an initiator selected from the group consisting of Azobis(isobutyronitril) (AlBN), 2,2′-Azodi(2-methylbutyronitrile) (VAZO 67), 1,1′-Azobis(cyanocyclohexane) (VAZO 88), benzoylperoxid (BPO), 2,2′-Azobis(2-amidinopropane) dihydrochloride (AAPH) and 4,4′-Azobis(4-cyanopentanoic acid) (ACVA)

Preferably, step (i-3) comprises:
(i-3-1) providing a composition (A) comprising the polymer precursor molecules according to (i-2), the at least one magnetic core (M) according to (i-1), at least one organic solvent, at least one initiator and a water phase, wherein the organic solvent is not miscible with water, and
(i-3-2) stirring composition (A) to give an emulsion (B), wherein the emulsion is preferably an organic solvent-in-water emulsion.

The monomers and the at least one magnetic core (M) are preferably suspended in a water solution optionally containing at least one suspending agent. The amount of water employed can vary widely, depending on the type of reactor employed, agitation means, and the like, though the final suspension mixture preferably contains about 5 to 60 percent by weight of the monomeric building blocks based on total weight of the entire mixture including water.

A variety of suspending agents can be employed as additives in suspension polymerization systems, since the method involves a liquid-in-liquid dispersion and affords a final product in the form of discrete solid particles. The suspension agents include insoluble carbonates, silicates, talc, gelatine, pectin, starch, cellulose derivatives, insoluble phosphates, PVA, salts, NaCl, KCl, PVP and the like. Preferably, the polymerization in (i-3) is carried out in the absence of any tensides.

The time employed for polymerization should be that sufficient for the degree or extent of conversion desired, and can vary over a wide range, depending on various reaction parameters such as the temperature employed, from a very few minutes to many hours, such as 48 hours.

Preferably, step (i-3) is carried out for a time in the range of from 1 hour to 30 hours, preferably 1 hour to 8 hours.

Temperatures employed are at least sufficient to effectuate thermal polymerization, or to cause decomposition of the free radical initiator, where used, which provides initiation of the reaction, preferably below temperatures which might cause gel formation of the polymer. Temperatures preferably employed are in the range of about 0° C. to 100° C., preferably 40 to 90° C.

The stirring is preferably carried out with an overhead stirrer.

### Step (ii)

In step (ii), a molecule comprising at least two amine groups within its structure or a molecule comprising at least two functional groups reactive towards amine groups is provided.

In a first embodiment of the inventive method, the molecule comprising at least two nitrogen atoms within its structure according to (ii) has the general structure of formula II

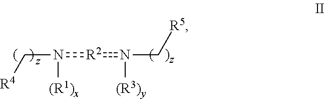

wherein:
x, y are independently 1 or 2;
z is zero or 1;
R¹, R³ are independently selected from the group consisting of hydrogen, C1-C10-alkyl, C1-C10-alkenyl, C5-C10-cycloalkyl, C5-C12-aryl, C4-C10-heteroaryl and —(—O—CH₂—CH₂—)ₙ—O—CH₃ with n being an integer in the range of from 1 to 15, wherein each of R¹, R³ may have at least one further substituent selected from the group consisting of hydrogen, C1-C5-alkyl, C5-C12-aryl, C4-C10-heteroaryl, and wherein R¹ and R³ are separate or together form an aliphatic or aromatic ring system;
R² is selected from the group consisting of C1-C10-alkyl, which is optionally substituted with a —COOH or COO⁻ group, C1-C10-alkenyl, C5-C10-cycloalkyl, C5-C12-aryl, C4-C10-heteroaryl and —(—O—CH₂—CH₂—)ₙ—O— with n being an integer in the range of from 1 to 15, wherein each cyclic structure having two or more ring systems has separated or annulated ring systems;
R⁴, R⁵ are independently hydrogen or represent a free electron pair;
   wherein the bonds connecting R² with each nitrogen atom are independently selected from the group consisting of single, double and aromatic bond.

According to one embodiment, the residue R² is selected from the group consisting of C1-C10-alkyl, C1-C10-alkenyl, C5-C10-cycloalkyl, C5-C12-aryl, C4-C10-heteroaryl and —(—O—CH₂—CH₂—)ₙ—O— with n being an integer in the range of from 1 to 15, wherein each cyclic structure having two or more ring systems has separated or annulated ring systems; wherein preferably each C1-C10-alkyl is not substituted with a carboxyl(ate) group, i.e. each C1-C10-alkyl has only hydrogen atoms as substituents at the carbon atoms.

Preferably, the molecule comprising at least two nitrogen atoms within its structure according to (ii) has the general structure of formula IIa

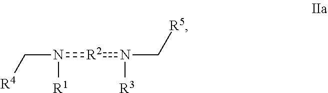

wherein R¹, R³ and R² together with the nitrogen atoms form an aromatic ring system comprising 3, 5, 7 or 9 carbon atoms, wherein the bonds connecting R² with each nitrogen atom are aromatic bonds; and R⁴, R⁵ are independently hydrogen or represent a free electron pair.

More preferably, the molecule comprising at least two nitrogen atoms within its structure according to (ii) having the general structure of formula IIa R² comprises one carbon atom and R¹, R³ together comprise 2, 4, 6 or 8 carbon atoms, wherein the bonds connecting R² with each nitrogen atom are aromatic bonds; and R⁴, R⁵ are independently hydrogen or represent a free electron pair.

More preferably, the molecule comprising at least two nitrogen atoms within its structure according to (ii) having the general structure of formula IIa is imidazole (IIa-1).

According to another preferred embodiment of the inventive method, the molecule comprising at least two nitrogen atoms within its structure according to (ii) has the general structure of formula IIb

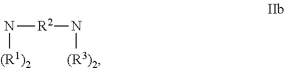

wherein R¹, R³ are independently selected from the group consisting of C1-C10-alkyl, C1-C10-alkenyl, and —(—O—CH₂—CH₂—)ₙ—O—CH₃ with n being an integer in the range of from 1 to 15, wherein each of R¹, R³ may have at least one further substituent selected from the group consisting of hydrogen, C1-C5-alkyl, C5-C12-aryl, C4-C10-heteroaryl, and wherein R¹ and R³ are separate; R² is selected from the group consisting of C1-C10-alkyl, C1-C10-alkenyl, and —(—O—CH₂—CH₂—)ₙ—O— with n being an integer in the range of from 1 to 15; wherein the bonds connecting R² with each nitrogen atom are single bonds.

More preferably, for the molecule comprising at least two nitrogen atoms within its structure according to (ii) having the general structure of formula IIb R¹, R³ are independently selected from the group consisting of C1-C10-alkyl, preferably from the group consisting of C1-C5-akyl; R² is selected from the group consisting of C1-C10-alkyl, preferably from the group consisting of C2-C8-akyl; wherein the bonds connecting R² with each nitrogen atom are single bonds.

More preferably, the molecule comprising at least two nitrogen atoms within its structure according to (ii) having the general structure of formula IIb is N,N,N′,N′-tetramethylethylenediamine (IIb-1).

According to another preferred embodiment of the inventive method, the molecule comprising at least two nitrogen atoms within its structure according to (ii) has the general structure of formula IIc:

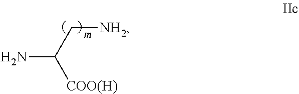

wherein m is an integer in the range of from 1 to 10, preferably in the range of from 2 to 8, more preferred in the range of from 3 to 6.

More preferably, the molecule comprising at least two nitrogen atoms within its structure according to (ii) having the general structure of formula IIc has the structure IIc-1:

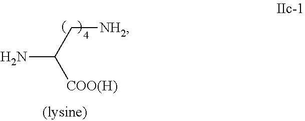

According to another preferred embodiment of the inventive method, the molecule comprising at least two nitrogen atoms within its structure according to (ii) has the general structure of formula a IId:

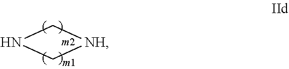

wherein m1 and m2 are independently integers in the range of from 2 to 10, preferably in the range of from 2 to 5.

More preferably, the molecule comprising at least two nitrogen atoms within its structure according to (ii) having the general structure of formula IId has the general structure of formula IId-1:

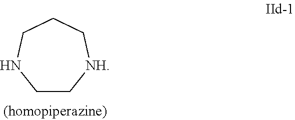

Step (iii)

In step (iii), the groups reactive to amine groups of the magnetic particle provided in (i) are reacted with the amine groups of the molecule provided in (ii) or the amine groups of the magnetic particle provided in (i) are reacted with the groups reactive towards amine groups of the molecule provided in (ii), thereby forming at least one hypercrosslinking bond; and thereby obtaining a hypercrosslinked magnetic particle.

Thus, the polymer matrix (P1) is hypercrosslinked in step (iii). Preferably, the reaction in (iii) is carried out at a temperature equal to or less than 200° C., more preferably in the range of from −80 to +200° C., more preferably in the range of from 20 to 100° C., more preferably in the range of from 50 to 95° C., more preferably in the range of from 70 to 90° C. Preferably, the reaction in (iii) is carried out for a reaction time in the range of from 0.01 to 200 h, more preferably in the range of from 0.1 to 200 h, preferably in the range of from 20 to 150 h, more preferred in the range of from 50 to 100 h. Preferably, the reaction in (iii) is carried out in a solvent (mixture), the solvent (mixture) comprising at least one solvent selected from the group of organic solvents, preferably from the group of non-halogenated organic solvents, more preferably from the group consisting of ethers, alcohols, aromatic organic solvents, acetonitrile, DMF, dioxane and DMSO, more preferably from the group consisting of isopropyl ether, diethylether, THF, ethanol, methanol, iso-propanol, n-propanol acetonitrile, DMF, dioxane and DMSO, more preferably from the group consisting of THF, acetonitrile, DMF, dioxane, toluene and DMSO.

In a further aspect, the present invention relates to a hypercrosslinked magnetic particle obtained or obtainable from the method as described above.

### Use/Method of Analyzing

According to a further aspect, the invention relates to the use of the hypercrosslinked magnetic particles as described above or of the hypercrosslinked magnetic particle obtained or obtainable from the method as described above for qualitative and/or quantitative determination of at least one analyte in a fluid or gas.

The term “qualitative” determination as used herein refers to determining the presence or absence of at least one analyte in the fluid or gas. Moreover, the term may also encompass an assessment of the nature of the analyte, i.e. it may encompass the identification of the analyte or the identification of a class of chemical molecules to which the analyte belongs.

The presence or absence of the at least one analyte can be determined by contacting the fluid sample or gas sample to the magnetic particles for a time and under conditions sufficient to allow for binding of the at least one analyte to the magnetic particle, subsequently removing the remaining fluid sample from the magnetic particle and determining whether the at least one analyte was bound to the magnetic particle, or not. In order to determine whether the analyte was bound to the magnetic particle, or not, compounds bound to the particle may be eluted by suitable techniques and the presence or absence of the at least one analyte may be subsequently determined in the eluate. Alternatively, the binding at least one analyte may be determined directly, i.e. bound to the magnetic particle.

The identification of the at least one analyte or the chemical class to which it belongs may be done after the said analyte has been eluted from the magnetic particle by suitable analytical methods such as mass spectrometry, UV-vis, NMR, IR or biochemical methods, such as ELISA, RIA and the like.

The term “quantitative” as used herein refers to determining the absolute or relative amount of the at least one analyte comprised in a fluid or gas sample.

The amount of the at least one analyte can be determined as described above for the qualitative determination. However, after elution of the analyte from the magnetic particles, the amount is to be determined in the eluate. Alternatively, the amount of bound analyte may be determined directly.

In light of the above, the present invention also contemplates a method for determining at least one analyte in a fluid or gas sample comprising the steps of:
(a) contacting a hypercrosslinked magnetic particle according to the invention or the hypercrosslinked magnetic particle obtained or obtainable by the method of the present invention with a fluid or gas sample comprising or suspected to comprise the at least one analyte; and
(b) determining the at least one analyte eluted from the said hypercrosslinked magnetic particle.

Typically, the determination referred to in this context is a qualitative or quantitative determination.

Typically, step (a) of the method is carried out for a time and under conditions sufficient to allow for binding of the at least one analyte to the magnetic particle. Thus, preferably in step (a) at least a portion, preferably all of, the analyte is bounded to the particle. In case, the determination is a quantitative determination, preferably substantially all of the anlalyte present in the fluid or gas sample is bound to the particle.

Preferably, step (a) further comprises step:
(a1) washing the hypercrosslinked magnetic particle to which at least a portion of the at least one analyte is bounded to, preferably under conditions which do not elute the at least one analyte; and/or
(a2) eluting the at least one analyte from the hypercrosslinked magnetic particle under conditions suitable to allow the elution of the at least one analyte.

More specifically, the qualitative or quantitative determination in (b) may comprise the determination of the presence or absence of bound analyte on the hypercrosslinked magnetic particle or the determination of the amount of analyte bound to the hypercrosslinked magnetic particle. It is to be understood that the washing step in (a1) may be carried as single washing step. Alternatively, more than one washing step may be carried out.

More specifically, the qualitative determination may comprise the following further step as part of step (a) and/or (b):
determining whether the at least one analyte was bound to the hypercrosslinked magnetic particle, or not.

The term fluid sample includes biological samples that have been manipulated in any way after their procurement, such as by treatment with reagents, solubilization, or enrichment for certain components, such as proteins or polynucleotides. Typically, the fluid sample is a liquid sample.

The term gas sample refers to a pure organic compound and mixtures of organic compounds, each in gaseous form. Depending on the boiling temperature of the compound(s) of interest, the gas may be formed by heating of a fluid and/or by lowering the pressure. Especially for compounds with low to intermediate boiling point (e.g. 20-100° C.) (semi)selective capturing by magnetic beads may be of interest. Also aerosols, although technically not gasses are here referred to as gaseous mixtures.

In a preferred embodiment the fluid sample as used herein refers to a biological sample obtained for the purpose of evaluation in vitro. In the methods of the present invention, the fluid sample or patient sample preferably may comprise any body fluid. In a preferred embodiment of the use as described above for qualitative and/or quantitative determination, the determination is an in vitro determination of an analyte in a body fluid sample of a mammal. Preferably, the body fluid sample is selected from the group consisting of blood, blood serum, urine, bile, stool, saliva, spinal fluid/liquid, plasma, re-dissolved dried blood spots and reconstituted dried samples of the aforementioned sample materials.

Depending on the nature of the fluid or gas sample, different classes of chemical compounds are to be detected. Preferably, the analyte in accordance with the present invention is selected from the group of organic compounds, preferably from the group consisting of steroids, sugars, vitamins, drugs, proteins, nucleic acids, sugars and mixtures of two or more thereof.

In a preferred embodiment of the use as described above for qualitative and/or quantitative determination, the determination is a qualitative and/or quantitative determination of an analyte in a plant sample.

The term plant sample refers to plant extracts. Magnetic particles that are able to capture compounds from these extracts may be useful for obtaining specific compounds, via a trap-and-elute mechanism, from these samples or removal of undesired compounds. Depending on the nature of the plant sample, different classes of chemical compounds are to be detected. Preferably, the analyte in accordance with the present invention is selected from the group of organic compounds, preferably from the group consisting of steroids, sugars, vitamins, drugs, proteins, nucleic acids, sugars and mixtures of two or more thereof.

The aforementioned applications for determining analytes in fluid or gas samples or in plant samples may, preferably, be applied or are involved in diagnostic purposes, drug of abuse testing, environmental control, food safety, quality control, purification or manufacturing processes. In diagnostic applications, the qualitative or quantitative determination of an analyte may allow aiding the diagnosis if the analyte is, e.g., a biomarker for a disease or medical condition. Similarly, the qualitative or quantitative assessment of an analyte being an indicator for environmental changes may help to identify pollution or to make assessments of environmental changes. Food safety as well as manufacturing or purification processes may be controlled by qualitative or quantitative determination of indicator analytes. Such indicators may also be determined in connection with general aspects of quality control, e.g., also in storage stability assessments of products and the like.

Preferably, the analyte is determined by mass spectrometry, UV-vis, NMR, IR.

According to a further aspect, the invention relates to the use of the hypercrosslinked magnetic particles as described above or of the hypercrosslinked magnetic particle obtained or obtainable from the method as described above for enrichment or purification of at least one analyte. Also here, the analyte in accordance with the present invention is selected from the group of organic compounds, preferably from the group consisting of steroids, sugars, vitamins, drugs, proteins, nucleic acids, sugars and mixtures of two or more thereof.

According to a further aspect, the invention relates to the use of the hypercrosslinked magnetic particles as described above or of the hypercrosslinked magnetic particle obtained or obtainable from the method as described above for purification of water, especially waste water. The term “purification” means that the content of at least one contamination is decreased in a water sample by treatment of the water sample with the hypercrosslinked magnetic particles according to the present invention. The contamination in accordance with the present invention is selected from the group of organic compounds, preferably from the group consisting of steroids, drugs, and drugs of abuse.

The present invention is further illustrated by the following embodiments and combinations of embodiments as indicated by the respective dependencies and back-references. In particular, it is noted that in each instance where a range of embodiments is mentioned, for example in the context of a term such as “The process of any one of embodiments 1 to 4”, every embodiment in this range is meant to be explicitly disclosed for the skilled person, i.e. the wording of this term is to be understood by the skilled person as being synonymous to “The process of any one of embodiments 1, 2, 3, and 4”.
1. Hypercrosslinked magnetic particle comprising a polymer matrix and at least one magnetic core (M), wherein the polymer matrix comprises at least one crosslinked polymer having at least one hypercrosslinking bond, wherein the hypercrosslinking bond consists of a molecule comprising at least two nitrogen atoms within its structure which are part of the hypercrosslinking bond; and having at least one positive charge;
wherein the molecule comprising at least two nitrogen atoms within its structure has the general structure of formula I

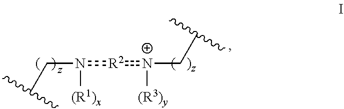

wherein
x, y are independently 1 or 2;
z is zero or 1;
R¹, R³ are independently selected from the group consisting of hydrogen, C1-C10-alkyl, C1-C10-alkenyl, C5-C10-cycloalkyl, C5-C12-aryl, C4-C10-heteroaryl and —(—O—CH₂—CH₂—)ₙ—O—CH₃ with n being an integer in the range of from 1 to 15, wherein each of R¹, R³ may have at least one further substituent selected from the group consisting of hydrogen, C1-C5-alkyl, C5-C12-aryl, C4-C10-heteroaryl, and wherein R¹ and R³ are separate or together with R² form an aliphatic or aromatic ring system;
R² is selected from the group consisting of C1-C10-alkyl, which is optionally substituted with a carboxyl(ate) group, C1-C10-alkenyl, C5-C10-cycloalkyl, C5-C12-aryl, C4-C10-heteroaryl and —(—O—CH₂—CH₂—)ₙ—O— with n being an integer in the range of from 1 to 15, wherein each cyclic structure having two or more ring systems has separated or annulated ring systems;
[symbol] the sinuous lines represent the crosslinked polymer;
wherein the bonds connecting R² with each nitrogen atom are independently selected from the group consisting of single, double and aromatic bond; and
wherein the molecule comprising at least two nitrogen atoms within its structure having the general structure of formula I has at least one positive charge.
2. Hypercrosslinked magnetic particle according to embodiment 1, wherein the magnetic particle has a particle size in the range of from 1 to 60 micrometer, as determined according to ISO 13320.
3. Hypercrosslinked magnetic particle according to embodiment 1 or 2, wherein the at least one positive charge of the molecule comprising at least two nitrogen atoms within its structure is compensated by at least one corresponding anion, the corresponding anion(s) being a carboxylate group of R² or being selected from the group consisting of F⁻, Cl⁻, Br⁻, I⁻, At⁻ and OH⁻, preferably selected from the group consisting of Cl⁻, Br⁻, I⁻ and OH⁻ and is more preferably OH⁻.
4. Hypercrosslinked magnetic particle according to any one of embodiments 1 to 3, wherein the molecule comprising at least two nitrogen atoms within its structure has the general structure of formula Ia:

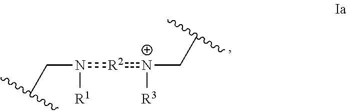

wherein the sinuous lines [symbol] represent the crosslinked polymer; R¹, R³ and R² together with the nitrogen atoms form an aromatic ring system comprising 3, 5, 7 or 9 carbon atoms;
wherein the bonds connecting R² with each nitrogen atom are aromatic bonds; and wherein the molecule has a positive charge, which is compensated by a corresponding anion, preferably OH⁻.
5. Hypercrosslinked magnetic particle according to embodiment 4, wherein in the molecule comprising at least two nitrogen atoms within its structure having the general structure of formula Ia R² comprises one carbon atom and R¹, R³ together comprise 2, 4, 6 or 8 carbon atoms, wherein the bonds connecting R² with each nitrogen atom are aromatic bonds; and wherein the molecule has a positive charge, which is compensated by a corresponding anion, preferably OH⁻.
6. Hypercrosslinked magnetic particle according to embodiment 5 or 6, wherein the molecule comprising at least two nitrogen atoms within its structure has the structure Ia-1:

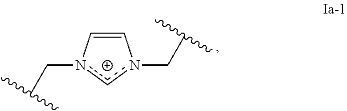

wherein the sinuous lines [symbol] represent the crosslinked polymer; and
wherein the positive charge is compensated by a corresponding anion, preferably OH⁻.
7. Hypercrosslinked magnetic particle according to any one of embodiments 1 to 3, wherein the molecule comprising at least two nitrogen atoms within its structure has the general structure of formula Ib:

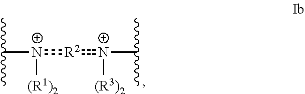

wherein
[symbol] the sinuous lines represent the crosslinked polymer;
R¹, R³ are independently selected from the group consisting of C1-C10-alkyl, C1-C10-alkenyl, and —(—O—CH₂—CH₂—)ₙ—O—CH₃ with n being an integer in the range of from 1 to 15, wherein each of R¹, R³ may have at least one further substituent selected from the group consisting of hydrogen, C1-C5-alkyl, C5-C12-aryl, C4-C10-heteroaryl, and wherein R¹ and R³ are separate;
R² is selected from the group consisting of C1-C10-alkyl, C1-C10-alkenyl, and —(—O—CH₂—CH₂—)ₙ—O— with n being an integer in the range of from 1 to 15;
wherein the bonds connecting R² with each nitrogen atom are single bonds; and wherein the molecule has two positive charges, which are compensated by corresponding anions, preferably OH⁻.
8. Hypercrosslinked magnetic particle according to embodiment 7, wherein in the molecule comprising at least two nitrogen atoms within its structure having the general structure of formula Ib:
   R¹, R³ are independently selected from the group consisting of C1-C10-alkyl, preferably from the group consisting of C1-C5-akyl;
   R² is selected from the group consisting of C1-C10-alkyl, preferably from the group consisting of C2-C8-akyl;
   wherein the bonds connecting R² with each nitrogen atom are single bonds; and wherein the molecule has two positive charges, which are compensated by corresponding anions, preferably OH⁻.
9. Hypercrosslinked magnetic particle according to any one of embodiments 7 or 8, wherein the molecule comprising at least two nitrogen atoms within its structure has the structure Ib-1:

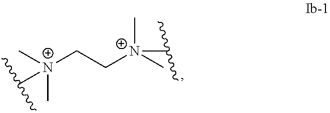 [SMILES: *[N+](C)(C)CC[N+](*)(C)C]

wherein the sinuous lines [symbol] represent the crosslinked polymer; and wherein the positive charges are compensated by corresponding anions, preferably OH⁻.
10. Hypercrosslinked magnetic particle according to any one of embodiments 1 to 3, wherein the molecule comprising at least two nitrogen atoms within its structure has the general structure of formula Ic:

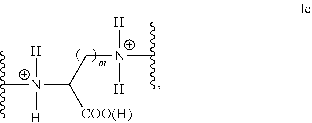

wherein
[symbol] the sinuous lines represent the crosslinked polymer; and m is an integer in the range of from 1 to 10, preferably in the range of from 2 to 8, more preferred in the range of from 3 to 6; wherein COO(H) represents a carboxyl(ate) group; and the molecule has two positive charges which are compensated by corresponding anions, preferably OH⁻.
11. Hypercrosslinked magnetic particle according to embodiment 10, wherein the molecule comprising at least two nitrogen atoms within its structure has the structure Ic-1:

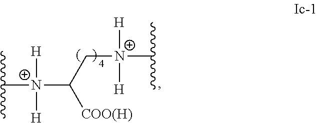

wherein the sinuous lines [symbol] represent the crosslinked polymer; and wherein the positive charges are compensated by corresponding anions, preferably OH⁻.
12. Hypercrosslinked magnetic particle according to any one of embodiments 1 to 3, wherein the molecule comprising at least two nitrogen atoms within its structure has the general structure of formula Id:

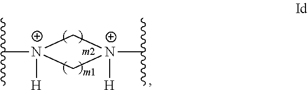

wherein the sinuous lines [symbol] represent the crosslinked polymer; m1 and m2 are independently integers in the range of from 2 to 10, preferably in the range of from 2 to 5; and wherein the molecule has two positive charges which are compensated by corresponding anions, preferably OH⁻.
13. Hypercrosslinked magnetic particle according to embodiment 12, wherein the molecule comprising at least two nitrogen atoms within its structure has the general structure of formula Id-1:

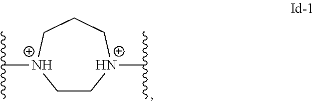 [SMILES: *[NH+]1CCC[NH+](*)CC1]

wherein the sinuous lines [symbol] represent the crosslinked polymer; and wherein the two positive charges are compensated by corresponding anions, preferably OH⁻.
14. Hypercrosslinked magnetic particle according to any one of embodiments 1 to 13, wherein the at least one magnetic core (M) comprises a compound selected from the group consisting of metal, metal carbide, metal nitride, metal sulfide, metal phosphide, metal oxide, metal chelate and a mixture of two or more thereof.
15. Hypercrosslinked magnetic particle according to any one of embodiments 1 to 14, wherein the at least one magnetic core (M) comprises a metal oxide or a metal carbide, more preferably, an iron oxide, in particular an iron oxide selected from the group consisting of Fe₃O₄, α-Fe₂O₃, γ-Fe₂O₃, MnFeₚO_{q}, CoFeₚO_{q}, NiFeₚO_{q}, CuFeₚO_{q}, ZnFeₚO_{q}—CdFeₚO_{q}, BaFeₚO and SrFeₚO, wherein p and q vary depending on the method of synthesis, and wherein p is preferably an integer of from 1 to 3, more preferably 2, and wherein q is preferably 3 or 4 most preferably, Fe₃O₄.
16. Hypercrosslinked magnetic particle according to any one of embodiments 1 to 15, wherein the at least one magnetic core (M) comprises at least one magnetic nanoparticle, preferably at least one iron oxide nanoparticle, more preferably a Fe₃O₄-nanoparticle.
17. Hypercrosslinked magnetic particle according to any one of embodiments 1 to 16, wherein the at least one magnetic core (M) comprises, more preferably consists of a magnetic nanoparticle and a coating C1.
18. Hypercrosslinked magnetic particle according to embodiment 17, wherein the coating C1 is selected from a group consisting of tensides, silica, silicates, silanes, phosphates, phosphonates, phosphonic acids and mixtures of two or more thereof, preferably the coating is a tenside coating.
19. A method of preparing a hypercrosslinked magnetic particle comprising a polymer matrix and at least one magnetic core (M), wherein the polymer matrix comprises at least one crosslinked polymer having at least one hypercrosslinking bond, wherein the hypercrosslinking bond consists of a molecule comprising at least two nitrogen atoms within its structure which are part of the hypercrosslinking bond, wherein the molecule comprising at least two nitrogen atoms within its structure has the general structure of formula I

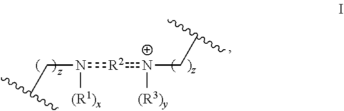

wherein
x, y are independently 1 or 2;
z is zero or 1;
R¹, R³ are independently selected from the group consisting of hydrogen, C1-C10-alkyl,
C1-C10-alkenyl, C5-C10-cycloalkyl, C5-C12-aryl, C4-C10-heteroaryl and —(—O—CH₂—CH₂—)ₙ—O—CH₃ with n being an integer in the range of from 1 to 15, wherein each of R¹, R³ may have at least one further substituent selected from the group consisting of hydrogen, C1-C5-alkyl, C5-C12-aryl, C4-C10-heteroaryl, and wherein R¹ and R³ are separate or together with R² form an aliphatic or aromatic ring system;
R² is selected from the group consisting of C1-C10-alkyl, which is optionally substituted with a —COOH or COO⁻ group, C1-C10-alkenyl, C5-C10-cycloalkyl, C5-C12-aryl, C4-C10-heteroaryl and —(—O—CH₂—CH₂—)ₙ—O— with n being an integer in the range of from 1 to 15, wherein each cyclic structure having two or more ring systems has separated or annulated ring systems;
[symbol] the sinuous lines represent the crosslinked polymer;
wherein the bonds connecting R² with each nitrogen atom are independently selected from the group consisting of single, double and aromatic bond; and
wherein the molecule comprising at least two nitrogen atoms within its structure having the general structure of formula I has at least one positive charge;
the method comprising:
   (iv) Providing a magnetic particle comprising a polymer matrix and at least one magnetic core (M), wherein the polymer matrix comprises, preferably consists of, at least one crosslinked polymer, which comprises at least two functional groups reactive towards amine groups;
   (v) providing a molecule comprising at least two amine groups within its structure;
   (vi) reacting the groups reactive to amine groups of the magnetic particle provided in (i) with the amine groups of the molecule provided in (ii) thereby forming at least one hypercrosslinking bond; and thereby obtaining a hypercrosslinked magnetic particle;
wherein the molecule comprising at least two nitrogen atoms within its structure according to (ii) has the general structure of formula II

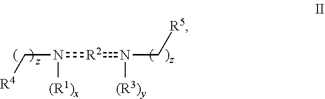

wherein:
x, y are independently 1 or 2;
z is zero or 1;
R¹, R³ are independently selected from the group consisting of hydrogen, C1-C10-alkyl, C1-C10-alkenyl, C5-C10-cycloalkyl, C5-C12-aryl, C4-C10-heteroaryl and —(—O—CH₂—CH₂—)ₙ—O—CH₃ with n being an integer in the range of from 1 to 15, wherein each of R¹, R³ may have at least one further substituent selected from the group consisting of hydrogen, C1-C5-alkyl, C5-C12-aryl, C4-C10-heteroaryl, and wherein R¹ and R³ are separate or together form an aliphatic or aromatic ring system;
R² is selected from the group consisting of C1-C10-alkyl, which is optionally substituted with a —COOH or COO⁻ group, C1-C10-alkenyl, C5-C10-cycloalkyl, C5-C12-aryl, C4-C10-heteroaryl and —(—O—CH₂—CH₂—)ₙ—O— with n being an integer in the range of from 1 to 15, wherein each cyclic structure having two or more ring systems has separated or annulated ring systems;
R⁴, R⁵ are independently hydrogen or represent a free electron pair;
wherein the bonds connecting R² with each nitrogen atom are independently selected from the group consisting of single, double and aromatic bond.
20. The method according to embodiment 19, wherein for the molecule comprising at least two nitrogen atoms within its structure according to (ii) has the general structure of formula IIa

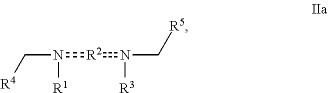

wherein R¹, R³ and R² together with the nitrogen atoms form an aromatic ring system comprising 3, 5, 7 or 9 carbon atoms, wherein the bonds connecting R² with each nitrogen atom are aromatic bonds; and R⁴, R⁵ are independently hydrogen or represent a free electron pair.
21. The method according to embodiment 20, wherein for the molecule comprising at least two nitrogen atoms within its structure according to (ii) having the general structure of formula IIa R² comprises one carbon atom and R¹, R³ together comprise 2, 4, 6 or 8 carbon atoms, wherein the bonds connecting R² with each nitrogen atom are aromatic bonds; and R⁴, R⁵ are independently hydrogen or represent a free electron pair.
22. The method according to embodiment 21, wherein the molecule comprising at least two nitrogen atoms within its structure according to (ii) having the general structure of formula IIa is imidazole (IIa-1).
23. The method according to embodiment 19, wherein the molecule comprising at least two nitrogen atoms within its structure according to (ii) has the general structure of formula IIb

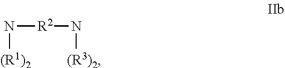

wherein R¹, R³ are independently selected from the group consisting of C1-C10-alkyl, C1-C10-alkenyl, and —(—O—CH₂—CH₂—)ₙ—O—CH₃ with n being an integer in the range of from 1 to 15, wherein each of R¹, R³ may have at least one further substituent selected from the group consisting of hydrogen, C1-C5-alkyl, C5-C12-aryl, C4-C10-heteroaryl, and wherein R¹ and R³ are separate; R² is selected from the group consisting of C1-C10-alkyl, C1-C10-alkenyl, and —(—O—CH₂—CH₂—)ₙ—O— with n being an integer in the range of from 1 to 15;
wherein the bonds connecting R² with each nitrogen atom are single bonds.
24. The method according to embodiment 23, wherein for the molecule comprising at least two nitrogen atoms within its structure according to (ii) having the general structure of formula IIb R¹, R³ are independently selected from the group consisting of C1-C10-alkyl, preferably from the group consisting of C1-C5-akyl; R² is selected from the group consisting of C1-C10-alkyl, preferably from the group consisting of C2-C8-akyl; wherein the bonds connecting R² with each nitrogen atom are single bonds.
25. The method according to embodiment 23 or 24, wherein the molecule comprising at least two nitrogen atoms within its structure according to (ii) having the general structure of formula IIb is N,N,N′,N′-tetramethylethylenediamine (IIb-1).
26. The method according to embodiment 19, wherein the molecule comprising at least two nitrogen atoms within its structure according to (ii) has the general structure of formula IIc:

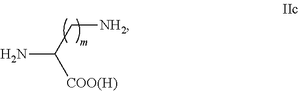

wherein m is an integer in the range of from 1 to 10, preferably in the range of from 2 to 8, more preferred in the range of from 3 to 6.
27. The method according to embodiment 26, wherein the molecule comprising at least two nitrogen atoms within its structure according to (ii) has the structure IIc-1:

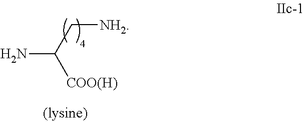

28. The method according to embodiment 19, wherein the molecule comprising at least two nitrogen atoms within its structure according to (ii) has the general structure of formula IId:

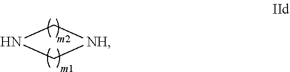

wherein m1 and m2 are independently integers in the range of from 2 to 10, preferably in the range of from 2 to 5.
29. The method according to embodiment 28, wherein the molecule comprising at least two nitrogen atoms within its structure according to (ii) has the general structure of formula IId-1:

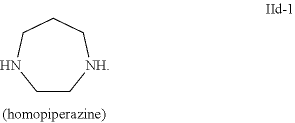

30. The method according to any one of embodiments 19 to 29, wherein (i) comprises:
   (i-1) providing at least one magnetic core (M);
   (i-2) providing polymer precursor molecules comprising at least one kind of polymer precursor molecules having functional groups reactive towards amine groups or amine groups,
   (i-3) polymerizing the polymer precursor molecules according to (i-2) in the presence of the at least one magnetic core (M), thereby forming a particle comprising the at least one magnetic core (M), embedded in a polymer matrix (P1), wherein the polymer matric (P1) preferably comprises, more preferably consists of, a crosslinked polymer having at least two functional groups reactive towards amine groups or at least two amine groups.
31. The method according to any one of embodiments 19 to 30, wherein the functional group reactive towards amine groups is selected from the group of halogenated C1-C3-alkyl group, halogen atom, epoxy group and activated carboxy group, preferably -acid halide or acid anhydride or succinimide.
32. The method according to any one of embodiments 19 to 31, wherein the reaction in (ii) is carried out at a temperature equal to or less than 200° C., preferably in the range of from −80 to +200° C., more preferably in the range of from 20 to 100° C., more preferred in the range of from 50 to 95° C., more preferred in the range of from 70 to 90° C.
33. The method according to any one of embodiments 19 to 32, wherein the reaction in (ii) is carried out for a reaction time in the range of from 0.01 to 200 h, preferably in the range of from 0.1 to 200 h, preferably in the range of from 20 to 150 h, more preferred in the range of from 50 to 100 h.
34. The method according to any one of embodiments 19 to 33, wherein the reaction in (ii) is carried out in a solvent (mixture), the solvent (mixture) comprising at least one solvent selected from the group of organic solvents, preferably from the group of non-halogenated organic solvents, more preferably from the group consisting of ethers, alcohols, aromatic organic solvents, acetonitrile, DMF, dioxane and DMSO, more preferably from the group consisting of isopropyl ether, diethylether, THF, ethanol, methanol, iso-propanol, n-propanol acetonitrile, DMF, dioxane and DMSO, more preferably from the group consisting of THF, acetonitrile, DMF, dioxane, toluene and DMSO.
35. The method according to any one of embodiments 19 to 34, wherein the polymerization in (i-3) is a suspension polymerization.
36. The method according to any one of embodiments 19 to 35, wherein the polymerization in (i-3) is carried out in the presence of an initiator selected from the group consisting of Azobis(isobutyronitril) (AlBN), 2,2′-Azodi(2-methylbutyronitrile) (VAZO 67), 1,1′-Azobis(cyanocyclohexane) (VAZO 88), benzoylperoxid (BPO), 2,2′-Azobis(2-amidinopropane) dihydrochloride (AAPH) and 4,4′-Azobis(4-cyanopentanoic acid) (ACVA)
37. The method according to any one of embodiments 19 to 36, wherein step (i-3) comprises:
   (i-3-1) providing a composition (A) comprising the polymer precursor molecules according to (i-2), the at least one magnetic core (M) according to (i-1), at least one organic solvent, at least one initiator and a water phase, wherein the organic solvent is not miscible with water, and
   (i-3-2) stirring composition (A) to give an emulsion (B), wherein the emulsion is preferably an organic solvent-in-water emulsion.
38. The method according to any one of embodiments 19 to 37, wherein the at least one magnetic core (M) comprises a compound selected from the group consisting of metal, metal carbide, metal nitride, metal sulfide, metal phosphide, metal oxide, metal chelate and a mixture of two or more thereof.
39. The method according to any one of embodiments 19 to 38, wherein the at least one magnetic core (M) comprises a metal oxide or a metal carbide, more preferably, an iron oxide, in particular an iron oxide selected from the group consisting of Fe₃O₄, α-Fe₂O₃, γ-Fe₂O₃, MnFeₚO_{q}, CoFeₚO_{q}, NiFeₚO_{q}, CuFeₚO_{q}, ZnFeₚO_{q}—CdFeₚO_{q}, BaFeₚO and SrFeₚO, wherein p and q vary depending on the method of synthesis, and wherein p is preferably an integer of from 1 to 3, more preferably 2, and wherein q is preferably 3 or 4 most preferably, Fe₃O₄.
40. The method of any one of embodiments 19 to 39, wherein the at least one magnetic core (M) comprises at least one magnetic nanoparticle, preferably at least one iron oxide nanoparticle, more preferably a Fe₃O₄-nanoparticle.
41. The method according to any one of embodiments 19 to 40, wherein step (i-1) comprises:
   (i-1.1) providing at least one magnetic nanoparticle, and
   (i-1.2) coating the at least one nanoparticle with a coating C1, the coating C1 preferably being selected from a group consisting of tensides, silica, silicates, silanes, phosphates, phosphonates, phosphonic acids and mixtures of two or more thereof
   to give the magnetic core (M).
42. Hypercrosslinked magnetic particle obtained or obtainable from the method according to any one of embodiments 19 to 41.
43. Use of the hypercrosslinked magnetic particles according to any one of embodiments 1 to 18 or of the hypercrosslinked magnetic particle obtained or obtainable from the method according to any one of embodiments 19 to 41 for qualitative and/or quantitative determination of at least one analyte in a fluid or gas.
44. The use according to embodiment 43 for qualitative and/or quantitative in vitro determination of an analyte in a body fluid sample of a mammal.
45. The use according to embodiment 44, wherein the body fluid sample is selected from the group consisting of blood, blood serum, urine, bile, stool, saliva, spinal fluid/liquid, plasma, re-dissolved dried blood spots and reconstituted dried samples of the aforementioned sample materials.
46. The use according to embodiment 43 for qualitative and/or quantitative determination of an analyte in a plant sample.
47. The use according to any one of embodiments 43 to 46, wherein the said analyte is selected from the group of organic compounds, preferably from the group consisting of steroids, sugars, vitamins, drugs, proteins, nucleic acids, sugars and mixtures of two or more thereof.
48. Use of the hypercrosslinked magnetic particles according to any one of embodiments 1 to 18 or of the hypercrosslinked magnetic particle obtained or obtainable from the method according to any one of embodiments 19 to 41 for enrichment or purification of at least one analyte.
49. The use according to any one of embodiments 43 to 48, wherein the said analyte is determined by mass spectrometry, UV-vis, NMR, IR.
50. Use of the hypercrosslinked magnetic particles according to any one of embodiments 1 to 18 or of the hypercrosslinked magnetic particle obtained or obtainable from the method according to any one of embodiments 19 to 41 for purification of water, especially waste water.

### EXAMPLES

The following Examples shall merely illustrate the invention. Whatsoever, they shall not be construed as limiting the scope of the invention.

### Example 1: Preparation of Hypercrosslinked Magnetic Polymer Particles (Beads)

### Tenside-Coated Magnetic Nanoparticles (1)

In a general procedure 126 g FeCl₂.4H₂O (0.63 mol) and 248 g FeCl₃ (1.53 mol) were added under stirring to 31 water and heated to 55° C. 460 ml NH₄OH (28% in H₂O) were added and after 15 min the black precipitate was separated with a magnet. The supernatant was discarded and the magnetic nanoparticles were washed three times with water. The magnetic nanoparticles were resuspended in 2000 ml and the pH was adjusted to 7-9 with NaOH (10 M). After ultrasonication for 30 min the suspension was transferred in a 4 l reactor and 1 l water was added. While stirring 120 ml oleic acid were added and the suspension was stirred for 45 min at 25° C. The magnetic nanoparticles were separated with a magnet and the supernatant was discarded. The tenside-coated nanoparticles were washed three times with water and ethanol and stored in ethanol to give tenside-coated magnetic nanoparticles (1) (203 g).

### Magnetic Polymer Particles (2)

In a general procedure 650 ml water was added to a 2 l glass reactor with mechanical stirrer. 13.5 g PVA was added and stirred until complete solution. 10 g of (1) were separated with a magnet and the supernatant was discarded. The magnetic nanoparticles were washed one time with toluene and then resuspended in 168 ml toluene. 23.6 ml divinylbenzene (0.17 mol) and 23.6 ml vinylbenzyl chloride (0.17 mol) were added and the mixture was ultrasonicated for 1 h. 3.84 g 2,2′-azobis(2-methylbutyronitrile) (20 mmol) were added and the mixture was transferred to the PVA-solution under rapid stirring. The mixture was heated to 80° C. and the reaction continued for 4 h. The formed magnetic polymer particles were separated with a magnet and the supernatant was discarded. The particles were washed three times with water and methanol and resuspended in isopropanol/water (10/90 v/v) to give magnetic polymer particles (2) (52.3 g).

### Porous Magnetic Polymer Particles/Diamine Hypercrosslinked Magnetic Polymer Particles (3)

In a general procedure 5 g of (2) were separated with a magnet and the supernatant was discarded. The magnetic polymer particles (2) were washed three times with CH₃CN and then resuspended in 150 ml CH₃CN. 2.8 g KOH (50 mmol) and 88 mmol of the chosen diamine imidazole were added under stirring. The suspension was heated to 80° C. and proceeded at this temperature for 72 h. Afterwards the particles were separated with a magnet and the supernatant was discarded. The particles were washed three times with ethanol and six times with water to give imidazole hypercrosslinked magnetic polymer particles (3a) (yield: 95%, 4.8 g). The same procedure was carried out with
TMEDA as the chosen diamine to give TMEDA hypercrosslinked magnetic polymer particles (3b) (96%);
Lysine as the chosen diamine to give lysine hypercrosslinked magnetic polymer particles (3d) (96%); and
Homopiperazine as the chosen diamine to give homopiperazine hypercrosslinked magnetic polymer particles (3e) (96%)

### Comparative—Porous Magnetic Polymer Particles/Friedel Crafts Hypercrosslinked Magnetic Polymer Particles (3c)

In a general procedure 5 g of (2) were separated with a magnet and the supernatant was discarded. The magnetic nanoparticles were washed three times with the hypercrosslinking solvent (dichloroethane, toluene, DMF, CH₃CN, dioxane or THF) and then resuspended in 60 ml in the chosen solvent. The suspension was stirred for 30 min and then heated to 80° C. When the temperature was reached the catalyst (FeCl₃ or ZnCl₂; 12 mmol) was added and nitrogen was bubbled through the suspension. After 1 h the particles were separated with a magnet and the supernatant was discarded. The particles were washed five times with ethanol to give Friedel Crafts (FC) hypercrosslinked porous magnetic polymer particles (3c) (4.8 g).

### Example 2: Evaluation of Hypercrosslinked Porous Magnetic Polymer Particles Based on TMEDA, Imidazole and Comparative FC Hypercrosslinked Porous Magnetic Polymer Particles

Synthesized hypercrosslinked porous magnetic polymer particles (3a), (3b), (3c), were evaluated for their propensity to capture and elute analytes.

### Analyte Capturing

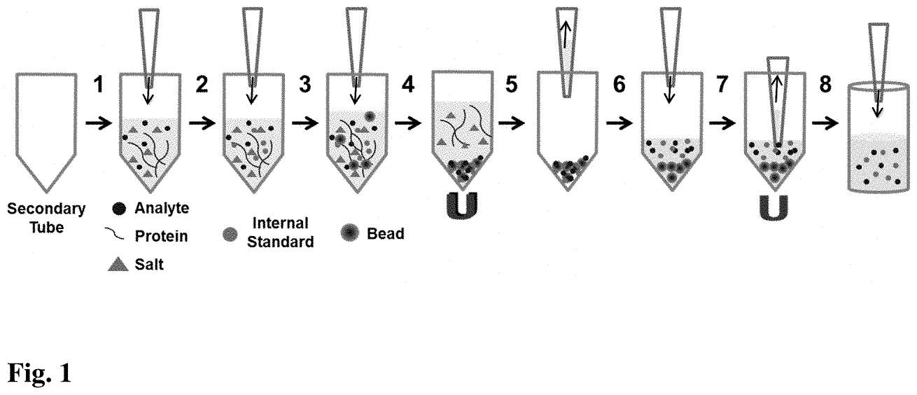

For the hypercrosslinked porous magnetic polymer particles (beads) evaluation the beads underwent the enrichment workflow as illustrated in FIG. 1. As test samples a spiked serum pool was used, where the spiked analytes are listed in the following Table 1.

TABLE 1
Analytes evaluated

| Analyte | Concentration (ng/mL) |
|---|---|
| Methamphetamine | 50 |
| Secobarbital | 700 |
| Clonazepam | 80 |
| Diazepam | 50 |
| BenzoylecgoninE | 5 |
| 6-Acetylmorphine | 50 |
| Morphine-3-glucuronide | 200 |
| Phencyclidine | 3 |
| Amitriptyline | 20 |
| Nortriptyline | 20 |
| Δ9 COOH-THC | 100 |
| EDDP | 5 |
| Aldosterone | 300 |
| Estradiol | 100 |
| Testosterone | 15 |
| Carbamazepine | 15 |
| Gabapentin | 50 |
| Phenytoin | 50 |
| Valproic acid | 25000 |
| Vancomycin | 500 |
| 5-fluorouracil (5-FU) | 500 |

### Preparation of Samples

Samples were prepared by spiking the 22 analytes of interest from Table 1 into an analyte-free human serum pool. Internal standard solution was a methanol/water 50:50 v/v mixture containing isotope labelled analogues of the target analytes.

### Bead Extraction

For each sample, 100 μL of spiked serum was mixed with 50 μL of bead suspension in water at a concentration of 50 mg/ml and equilibrated for 5 min at room temperature under gentle rolling conditions, so that the analytes could access the entire surface of the particles. The supernatant was then discarded and the magnetic beads washed twice with 200 μL of water. Elution took place with 100 μL acetonitrile/2% formic acid in water 70:30 v/v. In the next step, 40 μL eluate were withdrawn from the vial and transferred to an HPLC vial, where 40 μL internal standard solution was added prior to LC-MS/MS analysis.

### Recovery

Quantification was performed by external calibration. For this, a calibration curve was recorded in neat solution. Recovery was calculated by comparing the calculated concentration in the eluate fraction to the spiked amount.

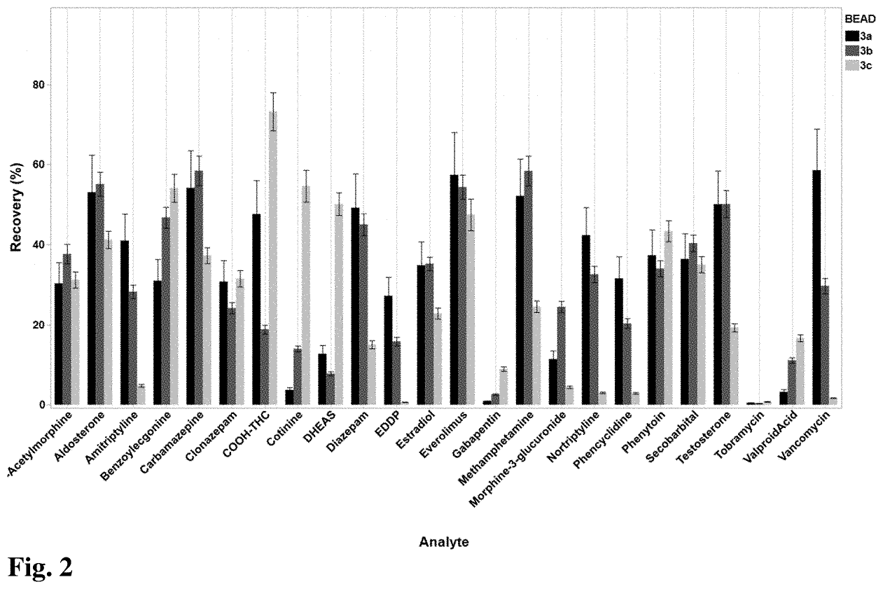

FIG. 2 depicts analyte recoveries that were obtained after sample preparation using the enrichment workflow as illustrated in FIG. 1. This shows that for many analytes both diamine hypercrosslinked porous magnetic polymer particles (3a) and (3b) perform better than the conventional FC-hypercrosslinked porous magnetic polymer particles (3c).

However, as it can be expected that different beads perform different under different workflows (i.e. with different organic solvents/pH settings/buffers) a better comparison would be to test the mentioned beads full factorial for each possible workflow combination. To reduce the workload, a DoE was performed whereby different factors were included. The eluates from the different workflows were then measured by means of LC-MS, whereby a number of analytes were quantified and their recovery after work-up was determined. From these data, using a linear model, predicted optimal recoveries were calculated. These predicted optimal recoveries are to be found in Table 2.

TABLE 2
Predicted optimal recoveries (%) for (3c), (3a) and
(3b). For the latter two, deviations in analyte recovery
vs. the recoveries for the FC-beads are shown.

| No. | Analyte | (3c) | (3a) | (3b) |
|---|---|---|---|---|
| 1 | Methamphetamine | 73 | 3 | −24 |
| 2 | Secobarbital | 75 | 0 | −11 |
| 3 | Clonazepam | 72 | 9 | 2 |
| 4 | Diazepam | 60 | −1 | −42 |
| 5 | Benzoylecgonine | 70 | −15 | −20 |
| 6 | 6-Acetylmorphine | 75 | 3 | −40 |
| 7 | Morphine-3-glucuronide | 36 | −10 | −13 |
| 8 | Phencyclidine | 73 | 26 | −69 |
| 9 | Amitriptyline | 76 | 5 | −37 |
| 10 | Nortriptyline | 75 | 7 | −37 |
| 11 | COOH-THC | 64 | −17 | 0 |
| 12 | EDDP | 70 | −1 | −46 |
| 13 | Aldosterone | 82 | 31 | −22 |
| 14 | Estradiol | 67 | 1 | −21 |
| 15 | Testosterone | 67 | −3 | −37 |
| 16 | Carbamazepine | 94 | 2 | −20 |
| 17 | Gabapentin | 12 | −3 | 9 |
| 18 | Phenytoin | 77 | 0 | −10 |
| 19 | Valproid acid | 68 | −12 | −18 |
| 20 | Vancomycin | 52 | −8 | −4 |
| 21 | 5-FU | <1 | <1 | 12 |

Some analytes show clearly a higher recovery, whereas others are obviously not captured and/or eluted as efficiently as the classical FC-beads. In addition, there seems to be a large influence on analyte recovery by the choice of diamine that is used for hypercrosslinking. In general, a lower recovery for most analytes in case TMEDA is used for hypercrosslinking was observed. However, for the polar analytes gabapentin and 5-fluorouracil a higher recovery was found. Of note is that especially 5-fluorouracil has not been captured before by any other bead tested so far.

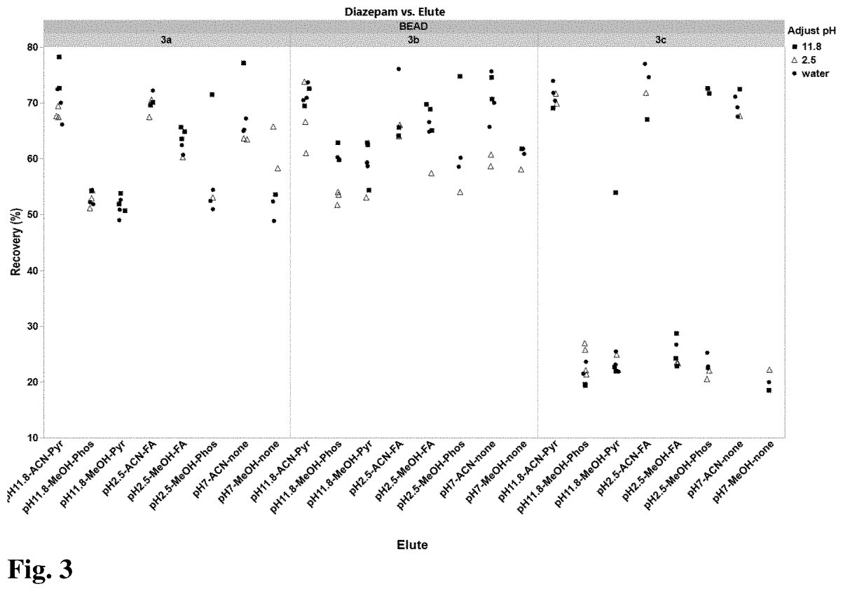

Furthermore, results from the same experiment show that diamine hypercrosslinked porous magnetic polymer particles were less sensitive to the choice of organic solvent that is used for elution of the analytes from the hypercrosslinked porous magnetic polymer particles. Two organic solvents were chosen for this purpose: MeOH and CH₃CN. This is depicted in FIG. 3.

Where FC-hypercrosslinked porous magnetic polymer particles showed higher recoveries when using CH₃CN, the diamine hypercrosslinked porous magnetic polymer particles were relatively indiscriminate towards either solvent. This was a clear advantage of diamine hypercrosslinked porous magnetic polymer particles as for the measurement by LC-MS is envisaged to operate with MeOH as mobile phase.

### Example 3: Evaluation of Hypercrosslinked Porous Magnetic Polymer Particles Based on on TMEDA, Imidazole, Lysine, Homopiperazine and Comparative FC Hypercrosslinked Porous Magnetic Polymer Particles

Synthesized hypercrosslinked porous magnetic polymer particles based on four different amines were evaluated for their propensity to capture and elute analytes, two crosslinked with a double tertiary amine (i.e. TMEDA (3b) and imidazole (3d)), one double secondary amine (i.e. homopiperazine (3d)), one with a double primary amine (i.e. lysine(3c)).

To test the propensity of these new beads to purify polar analytes from human serum, an analyte panel with clinically relevant analytes was combined and spiked in serum (see Table 3 below).

TABLE 3

| Analyte | Concentration (ng/ml) |
|---|---|
| Bezoylecgonine | 3 |
| Ethylglucuronide | 496 |
| Ethylsulfate | 545 |
| 2-oxo-3-hydroxy-LSD | 2 |
| Morphine-3-glucuronide | 100 |
| Gentamicin C1 | 297 |
| Gabapentin | 25 |
| Pregabalin | 496 |
| Theophylline | 99. |
| Amikacin | 297 |
| Tobramycin | 198 |
| Vancomycin | 24780 |
| 5-FU | 84 |
| Methotrexate | 20 |
| Ecgonine | 1090 |
| Mycophenolic acid | 5 |
| Buprenorphine glucuronide | 2.5 |
| Norbuprenorphine glucuronide | 2.5 |
| Noroxymorphone | 10 |

A standard bead-assessment workflow was as follows:

to the sample, to which analytes of interest have been spiked, a pH adjustment reagent was added that set the pH of the mixture (HCOOH or pyrrolidine or none). To this a bead suspension was added and the mixture was shaken and incubated for 5 min. Subsequently, a magnetic field was applied and the magnetic beads were drawn to the side of the vessel and the supernatant was removed. Next, a washing solution (water or buffer) was added and the mixture was shaken, after which the beads were again separated from the supernatant which was then again removed. This procedure was repeated once more. Subsequently, an elution solution was added and the mixture was shaken and incubated for another 5 min. Next, the beads were separated from the supernatant which was next transferred to another vial. To this, a mixture with the internal standards (ISTD-mix) of the compounds that have been spiked to the serum sample was added. Thus, no enrichment or dilution of the analytes was effected using this workflow. The procedure is summarized below in Table 4:

TABLE 4

| | pH | | | | | |
|---|---|---|---|---|---|---|
| Sample | Adjustment | Bead | Wash | Elution | Supernatant | Diluent |
| Serum | HCOOH/ | Bead | 2 times | 18 different | Transfer | ISTD-Mix |
| | pyrrolidine/ | suspension | with water | solvents/ | to vial | |
| | none | | or buffer | solutions | | |
| 100 μl | 40 μl | 40 μl | 150 μl | 50 μl | 30 μl | 30 μl |

Elution: Two different organic solvents were assessed: MeOH and CH₃CN, wherein three concentrations were used for each solvent: 0, 35 and 70 volume-% organic solvent, wherein 0 volume-% means that only water and buffer were used. For each concentration of each solvent three pH levels were set: pH 2.5 (100 mM HCOOH), pH 11.8 (100 mM pyrrolidine), pH 7 (non-buffered). Thus, 15 different elution solutions were used in total.

A set of factors and their ranges were defined. The use of a DoE allowed to compare a) new beads amongst each other, b) elution strength of acetonitrile vs methanol, at three different pH levels respectively (i.e. acidic, basic and neutral), c) the organic content of the elution solvent, d) pH adjustment of the serum/bead mixture. As mentioned before, the performance of the beads was directly influenced by other settings like pH, volumes, presence of organic solvents and its content. In order assess all these factors in combination with the beads, a full factorial DoE was carried out (for included factors see table 5 below), whereby the spiked human serum was worked up using the synthesized beads.

TABLE 5

| Step in workup protocol | Factors |
|---|---|
| pH Adjustment | 1) Water |
| | 2) 500 mM Pyrrolidine |
| | 3) 1M Formic acid |
| Washing | 1) Water |
| | 2) 25 mM Formic acid |
| | 3) 25 mM Pyrrolidine |
| Elution | Organic solvents: |
| | 1) MeOH |
| | 2) CH3CN |
| Elution | Organic content (% (v/v)): |
| | 1) 0 |
| | 2) 35 |
| | 3) 70 |
| Elution | pH Levels |
| | 1) 2.5 (using HCOOH) |
| | 2) 11.8 (using pyrrolidine) |
| | 3) 7 (no added buffer) |

The prepared samples were next measured using LC-MS/MS on an AB-Sciex 6500+ machine using electronspray as ion source. For integration and calculation of analyte concentration MultiQuant software tool was used. The DoE data were further analyzed using JMP SAS software.

The models could next be used to predict optimal recoveries of the assessed analytes. Below a graph is depicted the predicted optimal recoveries including 95% confidence intervals for each analyte, for each bead.

It was observed, that for some analytes higher recoveries could be obtained than compared to the conventional, Friedel-Crafts hypercrosslinked beads (3c). For the TMEDA hypercrosslinked bead (3b), methotrexate, 2-oxo-3-hydroxy LSD, benzoylecgonine, 5-fluorouracil and gabapentin showed an improvement in terms of recovery. The Homopiperazine bead (3e) showed better recoveries for methotrexate, tobramycin, 2-oxo-3-hydroxy LSD and ethylsulfate. The imidazole hypercrosslinked bead (3a) showed better recoveries for 2-oxo-3-hydroxy LSD, benzoylecgonine, gabapentin and ethylsulfate. The lysine hypercrosslinked bead (3d) was in several cases at least comparable to the Friedel-Crafts hypercrosslinked beads; for noroxymorphone the hypercrosslinked porous magnetic polymer particles based on lysine were a favourable alternative.

### Example 4: Evaluation of Hypercrosslinked Porous Magnetic Polymer Particles Based on on TMEDA and Comparative FC Hypercrosslinked Porous Magnetic Polymer Particles for an Enlarged Analyte Panel in Blood Serum and Urine

From experience (e.g. results from Example 3) it is known which sample preparation workflows would in principle yield the highest recoveries for each bead and each given analyte. To obtain absolute recoveries for each analyte for both the TMEDA- and the Friedel-Crafts hypercrosslinked beads under optimal conditions, an experiment was performed whereby 42 analytes were purified from urine and/or serum, using optimal workflows, in five replicates. The term optimal workflows in this case refers to settings like adjust pH, Elute pH, the number of wash cycles and the organic content of the elution that were found to be most optimal for each analyte-bead-sample type combination. These performed workflows in Table 6.

TABLE 6
workflows

| | | | Bead- | | | Organic |
|---|---|---|---|---|---|---|
| | Sample | pH | hypercross- | Wash | Elute | content |
| Analyte Name | Type | Adjust | linking | Number | pH | (volume-%) |
| 2-oxo-3- | Serum | 11 | TMEDA | 2 | 2.5 | 35 |
| hydroxy-LSD | | | | | | |
| 2-oxo-3- | Serum | 7 | FC | 2 | 2.5 | 70 |
| hydroxy-LSD | | | | | | |
| 2-oxo-3- | Urine | 11 | TMEDA | 2 | 2.5 | 70 |
| hydroxy-LSD | | | | | | |
| 2-oxo-3- | Urine | 11 | FC | 2 | 2.5 | 70 |
| hydroxy-LSD | | | | | | |
| 5-FU | Serum | 11 | TMEDA | 1 | 2.5 | 0 |
| 5-FU | Serum | 11 | FC | 2 | 2.5 | 90 |
| 5-FU | Urine | 11 | TMEDA | 1 | 2.5 | 0 |
| 5-FU | Urine | 7 | FC | 2 | 2.5 | 70 |
| 6-Acetylmorphine | Serum | 7 | TMEDA | 1 | 7 | 90 |
| 6-Acetylmorphine | Serum | 7 | FC | 1 | 7 | 90 |
| Aldosterone | Serum | 2.5 | TMEDA | 1 | 7 | 90 |
| Aldosterone | Serum | 7 | FC | 2 | 2.5 | 90 |
| Amikacin | Urine | 2.5 | TMEDA | 1 | 2.5 | 0 |
| Amikacin | Urine | 7 | FC | 2 | 2.5 | 0 |
| Amitriptyline | Serum | 2.5 | TMEDA | 2 | 2.5 | 90 |
| Amitriptyline | Serum | 7 | FC | 1 | 7 | 90 |
| Benzoylecgonine | Serum | 11 | TMEDA | 1 | 2.5 | 70 |
| Benzoylecgonine | Serum | 2.5 | FC | 2 | 7 | 70 |
| Benzoylecgonine | Urine | 11 | TMEDA | 1 | 2.5 | 0 |
| Benzoylecgonine | Urine | 2.5 | FC | 2 | 2.5 | 70 |
| Buprenorphine | Serum | 2.5 | TMEDA | 2 | 2.5 | 70 |
| glucuronide | | | | | | |
| Buprenorphine | Serum | 2.5 | FC | 2 | 11 | 70 |
| glucuronide | | | | | | |
| Buprenorphine | Urine | 11 | TMEDA | 2 | 2.5 | 70 |
| glucuronide | | | | | | |
| Buprenorphine | Urine | 11 | FC | 2 | 2.5 | 70 |
| glucuronide | | | | | | |
| Carbamazepine | Serum | 11 | TMEDA | 2 | 2.5 | 90 |
| Carbamazepine | Serum | 11 | FC | 2 | 2.5 | 90 |
| Clonazepam | Serum | 11 | TMEDA | 1 | 2.5 | 90 |
| Clonazepam | Serum | 2.5 | FC | 2 | 2.5 | 90 |
| Diazepam | Serum | 2.5 | TMEDA | 2 | 2.5 | 90 |
| Diazepam | Serum | 11 | FC | 2 | 2.5 | 90 |
| Ecgonine | Serum | 11 | TMEDA | 2 | 2.5 | 0 |
| Ecgonine | Serum | 2.5 | FC | 1 | 11 | 70 |
| Ecgonine | Urine | 2.5 | TMEDA | 1 | 2.5 | 0 |
| Ecgonine | Urine | 2.5 | FC | 2 | 11 | 70 |
| EDDP | Serum | 2.5 | TMEDA | 2 | 7 | 90 |
| EDDP | Serum | 7 | FC | 1 | 7 | 90 |
| Estradiol | Serum | 11 | TMEDA | 1 | 2.5 | 90 |
| Estradiol | Serum | 11 | FC | 2 | 2.5 | 90 |
| Ethyl glucuronide | Serum | 2.5 | TMEDA | 1 | 2.5 | 70 |
| Ethyl glucuronide | Serum | 11 | FC | 1 | 11 | 0 |
| Ethyl glucuronide | Urine | 2.5 | TMEDA | 2 | 2.5 | 0 |
| Ethyl glucuronide | Urine | 2.5 | FC | 2 | 2.5 | 0 |
| Ethylsulfate | Serum | 2.5 | TMEDA | 2 | 11 | 70 |
| Ethylsulfate | Serum | 2.5 | FC | 2 | 11 | 70 |
| Ethylsulfate | Urine | 2.5 | TMEDA | 2 | 11 | 70 |
| Ethylsulfate | Urine | 2.5 | FC | 2 | 11 | 70 |
| Fentanyl | Serum | 2.5 | TMEDA | 1 | 7 | 0 |
| Fentanyl | Serum | 2.5 | FC | 1 | 7 | 0 |
| Gabapentin | Serum | 11 | TMEDA | 2 | 2.5 | 0 |
| Gabapentin | Serum | 2.5 | FC | 1 | 11 | 90 |
| Hydrocodone | Urine | 2.5 | TMEDA | 1 | 11 | 0 |
| Hydrocodone | Urine | 2.5 | FC | 1 | 11 | 0 |
| Hydromorphone | Urine | 2.5 | TMEDA | 1 | 11 | 0 |
| Hydromorphone | Urine | 11 | FC | 2 | 2.5 | 70 |
| Ketamine | Serum | 11 | TMEDA | 1 | 11 | 70 |
| Ketamine | Serum | 11 | FC | 2 | 11 | 70 |
| Ketamine | Urine | 11 | TMEDA | 1 | 11 | 70 |
| Ketamine | Urine | 11 | FC | 2 | 11 | 70 |
| Levetiracetam | Serum | 11 | TMEDA | 1 | 11 | 70 |
| Levetiracetam | Serum | 11 | FC | 1 | 11 | 70 |
| Levetiracetam | Urine | 2.5 | TMEDA | 1 | 11 | 70 |
| Levetiracetam | Urine | 2.5 | FC | 1 | 11 | 70 |
| Methamphetamine | Serum | 11 | TMEDA | 2 | 11 | 90 |
| Methamphetamine | Serum | 11 | FC | 2 | 11 | 90 |
| Methotrexate | Serum | 2.5 | TMEDA | 2 | 2.5 | 70 |
| Methotrexate | Serum | 2.5 | FC | 2 | 2.5 | 70 |
| Methotrexate | Urine | 2.5 | TMEDA | 2 | 2.5 | 70 |
| Methotrexate | Urine | 2.5 | FC | 2 | 2.5 | 70 |
| Morphine-3- | Serum | 11 | TMEDA | 2 | 2.5 | 0 |
| glucuronide | | | | | | |
| Morphine-3- | Serum | 11 | FC | 1 | 2.5 | 90 |
| glucuronide | | | | | | |
| Morphine-3- | Urine | 11 | TMEDA | 1 | 2.5 | 0 |
| glucuronide | | | | | | |
| Morphine-3- | Urine | 11 | FC | 1 | 2.5 | 0 |
| glucuronide | | | | | | |
| Mycophenolic | Serum | 2.5 | TMEDA | 2 | 2.5 | 35 |
| acid | | | | | | |
| Mycophenolic | Serum | 11 | FC | 1 | 11 | 70 |
| acid | | | | | | |
| Mycophenolic | Urine | 11 | TMEDA | 2 | 2.5 | 70 |
| acid | | | | | | |
| Mycophenolic | Urine | 11 | FC | 2 | 2.5 | 70 |
| acid | | | | | | |
| Norbuprenorphine | Serum | 2.5 | TMEDA | 1 | 2.5 | 35 |
| glucuronide | | | | | | |
| Norbuprenorphine | Serum | 11 | FC | 2 | 11 | 70 |
| glucuronide | | | | | | |
| Norbuprenorphine | Urine | 11 | TMEDA | 2 | 2.5 | 70 |
| glucuronide | | | | | | |
| Norbuprenorphine | Urine | 11 | FC | 2 | 2.5 | 70 |
| glucuronide | | | | | | |
| Noroxymorphone | Serum | 11 | TMEDA | 2 | 2.5 | 0 |
| Noroxymorphone | Serum | 7 | FC | 2 | 2.5 | 0 |
| Noroxymorphone | Urine | 7 | TMEDA | 2 | 2.5 | 0 |
| Noroxymorphone | Urine | 7 | FC | 2 | 2.5 | 0 |
| Nortriptyline | Urine | 2.5 | TMEDA | 2 | 2.5 | 90 |
| Nortriptyline | Urine | 2.5 | FC | 1 | 11 | 90 |
| Oxycodone | Urine | 2.5 | TMEDA | 1 | 11 | 0 |
| Oxycodone | Urine | 2.5 | FC | 1 | 2.5 | 0 |
| Oxymorphone | Urine | 2.5 | TMEDA | 1 | 11 | 0 |
| Oxymorphone | Urine | 11 | FC | 2 | 2.5 | 70 |
| Phencyclidine | Serum | 7 | TMEDA | 1 | 7 | 90 |
| Phencyclidine | Serum | 7 | FC | 1 | 7 | 90 |
| Phenytoin | Serum | 2.5 | TMEDA | 1 | 2.5 | 90 |
| Phenytoin | Serum | 2.5 | FC | 2 | 2.5 | 90 |
| Pregabalin | Serum | 11 | TMEDA | 2 | 2.5 | 0 |
| Pregabalin | Serum | 11 | FC | 1 | 2.5 | 0 |
| Pregabalin | Urine | 11 | TMEDA | 2 | 2.5 | 0 |
| Pregabalin | Urine | 11 | FC | 2 | 2.5 | 0 |
| Secobarbital | Serum | 2.5 | TMEDA | 1 | 7 | 90 |
| Secobarbital | Serum | 2.5 | FC | 2 | 2.5 | 90 |
| Testosterone | Serum | 11 | TMEDA | 2 | 2.5 | 90 |
| Testosterone | Serum | 11 | FC | 2 | 2.5 | 90 |
| THC-COOH | Serum | 11 | TMEDA | 1 | 11 | 90 |
| THC-COOH | Serum | 11 | FC | 2 | 11 | 90 |
| Theophylline | Serum | 11 | TMEDA | 1 | 2.5 | 70 |
| Theophylline | Serum | 2.5 | FC | 1 | 11 | 0 |
| Tobramycin | Serum | 2.5 | TMEDA | 2 | 7 | 0 |
| Tobramycin | Serum | 2.5 | FC | 2 | 7 | 0 |
| Tobramycin | Urine | 11 | TMEDA | 2 | 11 | 35 |
| Tobramycin | Urine | 11 | FC | 2 | 11 | 70 |
| Tramadol | Urine | 2.5 | TMEDA | 1 | 11 | 0 |
| Tramadol | Urine | 2.5 | FC | 1 | 2.5 | 0 |
| Valproic acid | Serum | 2.5 | TMEDA | 2 | 2.5 | 90 |
| Valproic acid | Serum | 2.5 | FC | 2 | 2.5 | 90 |
| Vancomycin | Serum | 11 | TMEDA | 1 | 2.5 | 70 |
| Vancomycin | Serum | 11 | FC | 1 | 2.5 | 70 |
| Vancomycin | Urine | 11 | TMEDA | 1 | 2.5 | 35 |
| Vancomycin | Urine | 11 | FC | 2 | 2.5 | 70 |

Two analyte panels with clinically relevant analytes were combined and spiked in serum or urine (see Tables below).

TABLE 7
Analyte concentrations Serum

| | | Analyte Concentration |
|---|---|---|
| No. | Analyte | [ng/ml] |
| 1 | 2-oxo-3-hydroxy-LSD | 2 |
| 2 | 5-Fluorouracil | 85 |
| 3 | 6-Acetylmorphine | 25 |
| 4 | Aldosterone | 150 |
| 5 | Amikacin | 250 |
| 6 | Amitriptyline | 10 |
| 7 | Benzoylecgonine | 2.5 |
| 8 | Buprenorphine glucuronide | 2.5 |
| 9 | Carbamazepine | 7.5 |
| 10 | Clonazepam | 40 |
| 11 | Diazepam | 25 |
| 12 | Ecgonine | 100 |
| 13 | EDDP | 2.5 |
| 14 | Estradiol | 100 |
| 15 | Ethylglucuronide | 405 |
| 16 | Ethylsulfate | 61 |
| 17 | Gabapentin | 25 |
| 18 | Gentamicin | 400 |
| 19 | Ketamin | 10 |
| 20 | Levetiracetam | 200 |
| 21 | Methamphetamine | 25 |
| 22 | Methotrexate | 20 |
| 23 | Morphine-3-glucuronide | 100 |
| 24 | Mycophenolic acid | 5 |
| 25 | Norbuprenorphine glucuronide | 2.5 |
| 26 | Noroxymorphone | 10 |
| 27 | Nortriptyline | 10 |
| 28 | Phencyclidine | 1.5 |
| 29 | Phenytoin | 25 |
| 30 | Pregabalin | 250 |
| 31 | Secobarbital | 350 |
| 32 | Testosterone | 7.5 |
| 33 | Theophylline | 100 |
| 34 | Tobramycin | 200 |
| 35 | Valproic acid | 12500 |
| 36 | Vancomycin | 250 |
| 37 | Δ9 COOH-THC | 50 |

TABLE 8
Analyte concentrations Urine

| | | Analyte Concentration |
|---|---|---|
| No. | Analyte | [ng/ml] |
| 1 | 2-oxo-3-hydroxy-LSD | 2 |
| 2 | 5-Fluorouracil | 100 |
| 3 | Aldosterone | 10 |
| 4 | Benzoylecgonine | 2.5 |
| 5 | Buprenorphine glucuronide | 2.5 |
| 7 | Cortisone | 10 |
| 8 | Ecgonine | 100 |
| 9 | Ethylglucuronide | 3010 |
| 10 | Ethylsulfate | 487 |
| 11 | Fentanyl | 0.05 |
| 12 | Gabapentin | 25 |
| 13 | Hydrocodone | 10 |
| 14 | Hydromorphone | 10 |
| 15 | Ketamin | 10 |
| 16 | Levetiracetam | 200 |
| 17 | Methotrexate | 20 |
| 18 | morphine-3-glucuronide | 20 |
| 19 | Mycophenolic acid | 5 |
| 20 | Norbuprenorphine glucuronide | 2.5 |
| 21 | Noroxymorphone | 10 |
| 22 | Oxycodone | 10 |
| 23 | Oxymorphone | 10 |
| 24 | Pregabalin | 250 |
| 25 | Theophylline | 100 |
| 26 | Tobramycin | 200 |
| 27 | Tramadol | 50 |
| 28 | Vancomycin | 2500 |

### Methods

A standard bead-assessment workflow was as follows:

to the sample, to which analytes of interest have been spiked (vide infra for details), a pH adjustment reagent was added that sets the pH of the mixture. To this a bead suspension was added and the mixture was shaken and incubated for 5 min. Subsequently, a magnetic field was applied and the magnetic beads are drawn to the side of the vessel and the supernatant was removed. Next, a washing solution was added and the mixture was shaken, after which the beads were again separated from the supernatant which was then again removed. This procedure was repeated once more. Subsequently, an elution solution was added and the mixture was shaken and incubated for another 5 min. Next, the beads were separated from the supernatant which was next transferred to another vial. To this, a mixture with the internal standards of the compounds that have been spiked to the serum sample was added. Thus, no enrichment or dilution of the analytes was effected using this workflow.

TABLE 9

| | pH | | | | | |
|---|---|---|---|---|---|---|
| Sample | Adjustment | Bead | Wash | PT (EL) | Supernatant | Diluent |
| Serum | HCOOH/ | Bead | 1-2 times | Elution (CH₃CN | Transfer | ISTD- |
| | K₃PO₄—K₂HPO₄/ | suspension | with water | with Elute | to vial | Mix |
| | none | | | pH buffer) | | |
| 100 μL | 40 μL | 40 μL | 150 μL | 50 μL | 30 μL | 30 μL |

The prepared samples were next measured using LC-MS/MS on an AB-Sciex 6500+ machine using electrospray as ion source. For integration and calculation of analyte concentration MultiQuant software tool was used. Data were further analyzed using JMP SAS software.

### Results

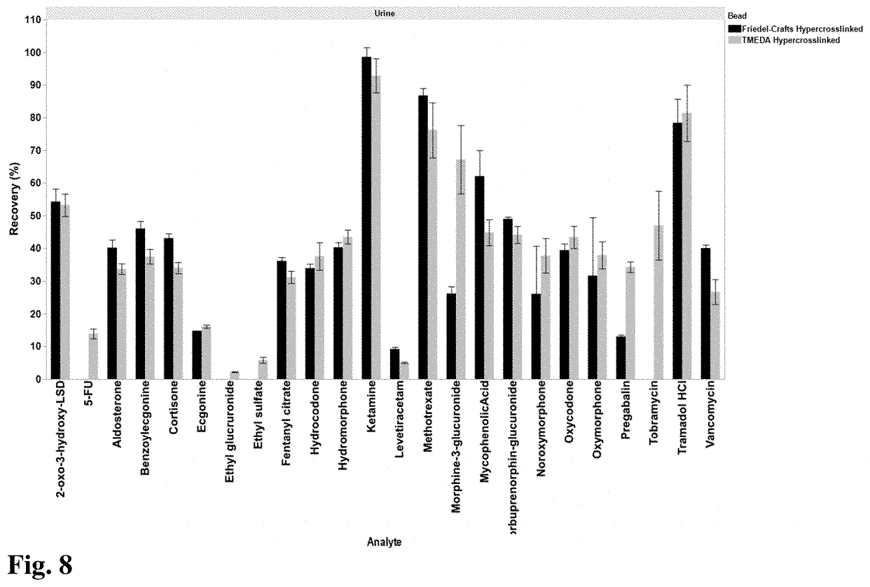

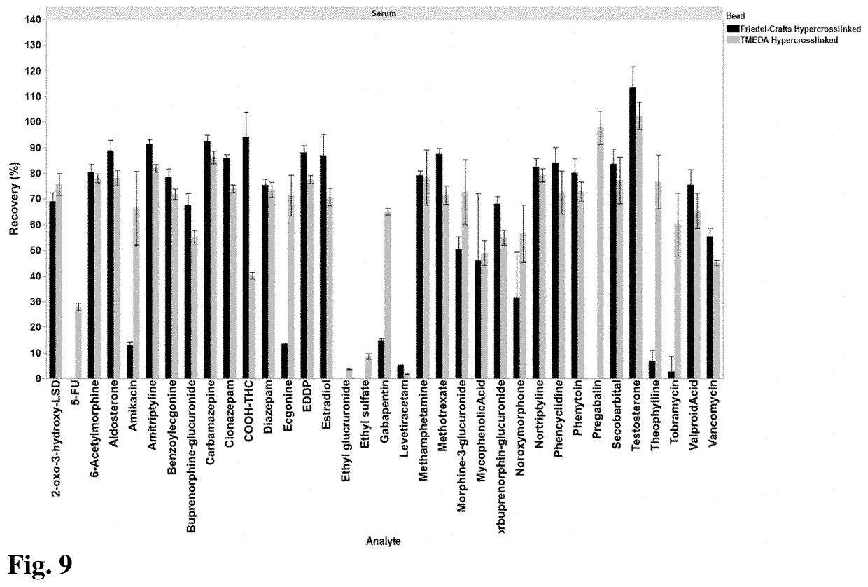

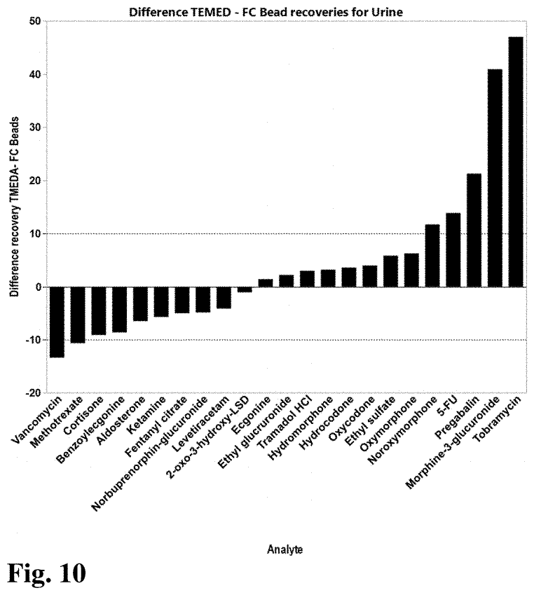

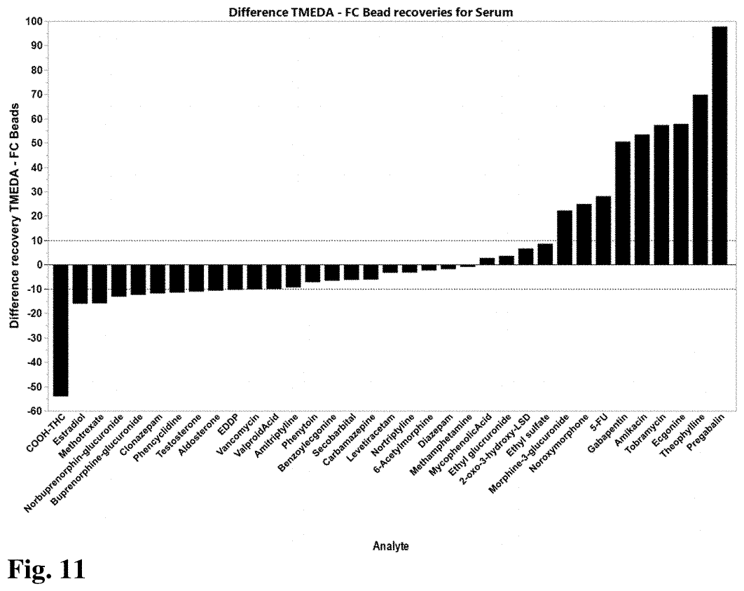

FIGS. 8 and 9 show the absolute recoveries for each analyte and each bead for urine and serum respectively. Urine data for buprenorphine-glucuronide and theophylline were removed as grave interferences from endogenous substances were encountered. From this it was noticed that for most analytes similar recoveries are obtained, regardless whether these substances are apolar for the most part. This applied to both analytes worked up from urine as well as serum. However, the difference between two beads was that for some analytes that are not or only to a low extend recovered from FC-hypercrosslinked beads, may yield recoveries that were acceptable to high when recovered via a workflow that makes use of the TMEDA bead. Notable examples for this are 5-FU, ethyl-glucuronide, pregabalin, ecgonine, ethyl sulfate, tobramycin, amikacin. The difference between two beads was further visualized in FIG. 10 (urine analytes) and FIG. 11 (serum analytes). Only in the case of THC—COOH we notice the advantage of the FC-bead, which yields ˜90% recovery, compared to about 40% recovery for the TMEDA bead. This suggests that the ionic-interaction between the carboxylic acid of this substance is less important than the hydrophobic interaction that this analyte may have to a higher degree with the FC-bead.

### CONCLUSIONS

Two important results were obtained: First, the TMEDA bead was shown once again to be able to yield high recoveries for most analytes from a chemically diverse analyte panel, both in urine as well as in serum. Secondly, it was shown that the TMEDA-hypercrosslinked covers a wider range of chemically diverse analytes that it is able to cover when compared to a bead that was hypercrosslinked via a Friedel-Crafts alkylation.

1. A hypercrosslinked magnetic particle comprising a polymer matrix and at least one magnetic core (M), wherein the polymer matrix comprises at least one crosslinked polymer having at least one hypercrosslinking bond, wherein the hypercrosslinking bond consists of a molecule comprising at least two nitrogen atoms within its structure which are part of the hypercrosslinking bond; and having at least one positive charge, wherein the molecule comprising at least two nitrogen atoms within its structure has the general structure of formula I 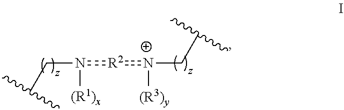
wherein
x, y are independently 1 or 2;
z is zero or 1; R¹, R³ are independently selected from the group consisting of hydrogen, C1-C10-alkyl, which is optionally substituted with a carboxyl(ate) group, C1-C10-alkenyl, C5-C10-cycloalkyl, C5-C12-aryl, C4-C10-heteroaryl and —(—O—CH₂—CH₂—)ₙ—O—CH₃ with n being an integer in the range of from 1 to 15, wherein each of R¹, R³ may have at least one further substituent selected from the group consisting of hydrogen, C1-C5-alkyl, C5-C12-aryl, C4-C10-heteroaryl, and wherein R¹ and R³ are separate or together with R² form an aliphatic or aromatic ring system;
R² is selected from the group consisting of C1-C10-alkyl, C1-C10-alkenyl, C5-C10-cycloalkyl, C5-C12-aryl, C4-C10-heteroaryl and —(—O—CH₂—CH₂—)ₙ—O— with n being an integer in the range of from 1 to 15, wherein each cyclic structure having two or more ring systems has separated or annulated ring systems; [symbol]
the sinuous lines represent the crosslinked polymer; wherein the bonds connecting R² with each nitrogen atom are independently selected from the group consisting of single, double and aromatic bond; and wherein the molecule comprising at least two nitrogen atoms within its structure having the general structure of formula I has at least one positive charge.

2. The hypercrosslinked magnetic particle according to claim 1, wherein the at least one positive charge of the molecule comprising at least two nitrogen atoms within its structure is compensated by at least one corresponding anion, the corresponding anion(s) being a carboxylate group of R² or being selected from the group consisting of F⁻, Cl⁻, Br⁻, I⁻, At⁻ and OH⁻.

3. The hypercrosslinked magnetic particle according to claim 1, wherein the molecule comprising at least two nitrogen atoms within its structure has the general structure of formula Ia: 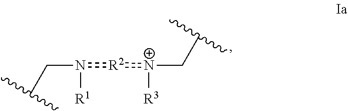
wherein the sinuous lines [symbol] represent the crosslinked polymer; R¹, R³ and R² together with the nitrogen atoms form an aromatic ring system comprising 3, 5, 7 or 9 carbon atoms; wherein the bonds connecting R² with each nitrogen atom are aromatic bonds; and wherein the molecule has a positive charge, which is compensated by a corresponding anion; wherein in the molecule comprising at least two nitrogen atoms within its structure having the general structure of formula Ia R² comprises one carbon atom and R¹, R³ together comprise 2, 4, 6 or 8 carbon atoms, wherein the bonds connecting R² with each nitrogen atom are aromatic bonds; and wherein the molecule has a positive charge, which is compensated by a corresponding anion.

4. The hypercrosslinked magnetic particle according to claim 1, wherein the molecule comprising at least two nitrogen atoms within its structure has the general structure of formula Ib: 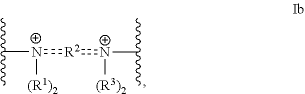
wherein [symbol]
the sinuous lines represent the crosslinked polymer;
R¹, R³ are independently selected from the group consisting of C1-C10-alkyl, C1-C10-alkenyl, and —(—O—CH₂—CH₂—)ₙ—O—CH₃ with n being an integer in the range of from 1 to 15, wherein each of R¹, R³ may have at least one further substituent selected from the group consisting of hydrogen, C1-C5-alkyl, C5-C12-aryl, C4-C10-heteroaryl, and wherein R¹ and R³ are separate;
R² is selected from the group consisting of C1-C10-alkyl, C1-C10-alkenyl, and —(—O—CH₂—CH₂—)ₙ—O— with n being an integer in the range of from 1 to 15;
wherein the bonds connecting R² with each nitrogen atom are single bonds; and wherein the molecule has two positive charges, which are compensated by corresponding anions;
wherein in the molecule comprising at least two nitrogen atoms within its structure having the general structure of formula Ib:
R¹, R³ are independently selected from the group consisting of C1-C10-alkyl;
R² is selected from the group consisting of C1-C10-alkyl;
wherein the bonds connecting R² with each nitrogen atom are single bonds; and wherein the molecule has two positive charges, which are compensated by corresponding anions.

5. The hypercrosslinked magnetic particle according to claim 1, wherein the molecule comprising at least two nitrogen atoms within its structure has the general structure of formula Ic: 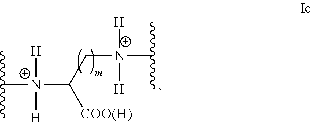
wherein [symbol]
the sinuous lines represent the crosslinked polymer; and m is an integer in the range of from 1 to 10; wherein COO(H) represents a carboxyl(ate) group; and the molecule has two positive charges which are compensated by corresponding anions.

6. The hypercrosslinked magnetic particle according to claim 1, wherein the molecule comprising at least two nitrogen atoms within its structure has the general structure of formula Id: 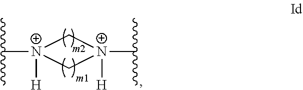
wherein the sinuous lines [symbol] represent the crosslinked polymer; m1 and m2 are independently integers in the range of from 2 to 10; and wherein the molecule has two positive charges which are compensated by corresponding anions.

7. A method of preparing a hypercrosslinked magnetic particle comprising a polymer matrix and at least one magnetic core (M), wherein the polymer matrix comprises at least one crosslinked polymer having at least one hypercrosslinking bond, wherein the hypercrosslinking bond consists of a molecule comprising at least two nitrogen atoms within its structure which are part of the hypercrosslinking bond, wherein the molecule comprising at least two nitrogen atoms within its structure has the general structure of formula I 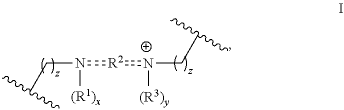
wherein
x, y are independently 1 or 2;
z is zero or 1;
R¹, R³ are independently selected from the group consisting of hydrogen, C1-C10-alkyl, C1-C10-alkenyl, C5-C10-cycloalkyl, C5-C12-aryl, C4-C10-heteroaryl and —(—O—CH₂—CH₂—)ₙ—O—CH₃ with n being an integer in the range of from 1 to 15, wherein each of R¹, R³ may have at least one further substituent selected from the group consisting of hydrogen, C1-C5-alkyl, C5-C12-aryl, C4-C10-heteroaryl, and wherein R¹ and R³ are separate or together with R² form an aliphatic or aromatic ring system;
R² is selected from the group consisting of C1-C10-alkyl, which is optionally substituted with a —COOH or COO⁻ group, C1-C10-alkenyl, C5-C10-cycloalkyl, C5-C12-aryl, C4-C10-heteroaryl and (—O—CH₂—CH₂—)ₙ—O— with n being an integer in the range of from 1 to 15, wherein each cyclic structure having two or more ring systems has separated or annulated ring systems; [symbol]
the sinuous lines represent the crosslinked polymer;
wherein the bonds connecting R² with each nitrogen atom are independently selected from the group consisting of single, double and aromatic bond; and
wherein the molecule comprising at least two nitrogen atoms within its structure having the general structure of formula I has at least one positive charge;
the method comprising:
(i) Providing a magnetic particle comprising a polymer matrix and at least one magnetic core (M), wherein the polymer matrix comprises at least one crosslinked polymer, which comprises at least two functional groups reactive towards amine groups;
(ii) providing a molecule comprising at least two amine groups within its structure;
(iii) reacting the groups reactive to amine groups of the magnetic particle provided in (i) with the amine groups of the molecule provided in (ii) thereby forming at least one hypercrosslinking bond; and thereby obtaining a hypercrosslinked magnetic particle,
wherein the molecule comprising at least two nitrogen atoms within its structure has the general structure of formula II 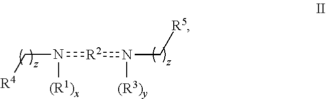
wherein
x, y are independently 1 or 2;
z is zero or 1; R¹, R³ are independently selected from the group consisting of hydrogen, C1-C10-alkyl, C1-C10-alkenyl, C5-C10-cycloalkyl, C5-C12-aryl, C4-C10-heteroaryl and —(—O—CH₂—CH₂—)ₙ—O—CH₃ with n being an integer in the range of from 1 to 15, wherein each of R¹, R³ may have at least one further substituent selected from the group consisting of hydrogen, C1-C5-alkyl, C5-C12-aryl, C4-C10-heteroaryl, and wherein R¹ and R³ are separate or together form an aliphatic or aromatic ring system;
R² is selected from the group consisting of C1-C10-alkyl, which is optionally substituted with a —COOH or COO⁻ group, C1-C10-alkenyl, C5-C10-cycloalkyl, C5-C12-aryl, C4-C10-heteroaryl and (—O—CH₂—CH₂—)ₙ—O— with n being an integer in the range of from 1 to 15, wherein each cyclic structure having two or more ring systems has separated or annulated ring systems;
R⁴, R⁵ are independently hydrogen or represent a free electron pair;
wherein the bonds connecting R² with each nitrogen atom are independently selected from the group consisting of single, double and aromatic bond.

8. The method according to claim 7, wherein for the molecule comprising at least two nitrogen atoms within its structure according to (ii) has the general structure of formula IIa 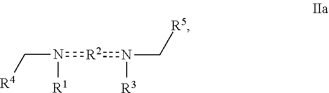
wherein R¹, R³ and R² together with the nitrogen atoms form an aromatic ring system comprising 3, 5, 7 or 9 carbon atoms, wherein the bonds connecting R² with each nitrogen atom are aromatic bonds; and R⁴, R⁵ are independently hydrogen or represent a free electron pair; wherein for the molecule comprising at least two nitrogen atoms within its structure having the general structure of formula IIa R² comprises one carbon atom and R¹, R³ together comprise 2, 4, 6 or 8 carbon atoms, wherein the bonds connecting R² with each nitrogen atom are aromatic bonds; and R⁴, R⁵ are independently hydrogen or represent a free electron pair, wherein the molecule comprising at least two nitrogen atoms within its structure having the general structure of formula IIa is imidazole (IIa-1).

9. The method according to claim 7, wherein for the molecule comprising at least two nitrogen atoms within its structure according to (ii) has the general structure of formula IIb 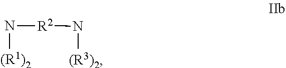
wherein R¹, R³ are independently selected from the group consisting of C1-C10-alkyl, C1-C10-alkenyl, and —(—O—CH₂—CH₂—)ₙ—O—CH₃ with n being an integer in the range of from 1 to 15, wherein each of R¹, R³ may optionally have at least one further substituent selected from the group consisting of hydrogen, C1-C5-alkyl, C5-C12-aryl, C4-C10-heteroaryl, and wherein R¹ and R³ are separate; R² is selected from the group consisting of C1-C10-alkyl, C1-C10-alkenyl, and —(—O—CH₂—CH₂—)ₙ—O— with n being an integer in the range of from 1 to 15; wherein the bonds connecting R² with each nitrogen atom are single bonds; wherein for the molecule comprising at least two nitrogen atoms within its structure having the general structure of formula IIb R¹, R³ are independently selected from the group consisting of C1-C10-alkyl; R² is selected from the group consisting of C1-C10-alkyl; wherein the bonds connecting R² with each nitrogen atom are single bonds, wherein the molecule comprising at least two nitrogen atoms within its structure having the general structure of formula IIb is N,N,N′,N′-tetramethylethylenediamine (IIb-1).

10. The method according to claim 7, wherein the molecule comprising at least two nitrogen atoms within its structure according to (ii) has the general structure of formula Tic: 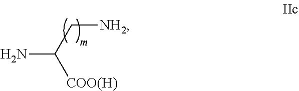
wherein m is an integer in the range of from 1 to 10; wherein the molecule comprising at least two nitrogen atoms within its structure having the general structure of formula IIc is lysine (IIc-1).

11. The method according to claim 7, wherein the molecule comprising at least two nitrogen atoms within its structure according to (ii) has the general structure of formula IId: 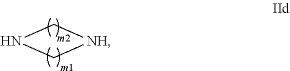
wherein m1 and m2 are independently integers in the range of from 2 to 10, wherein the molecule comprising at least two nitrogen atoms within its structure having the general structure of formula IId is homopiperazin (IId-1).
